(19) United States
(12) Patent Application Publication
(10) Pub. No.: US 2007/0015172 A1
(43) Pub. Date: Jan. 18, 2007

(54) Expression profiles for microbial infection

(75) Inventors: Hong Zhang, Germantown, MD (US); Yan A. Su, Bethesda, MD (US); Peisheng Hu, Covina, CA (US)

Correspondence Address: LAHIVE & COCKFIELD, 28 STATE STREET, BOSTON, MA, 02109, US

(73) Assignee: Z-BioMed, Inc., Rockville, MD (US)

(21) Appl. No.: 11/291,506

(22) Filed: Nov. 30, 2005

Related U.S. Application Data
(60) Provisional application No. 60/685,888, filed on Jun. 1, 2005.

(51) Int. Cl. C12Q 1/70; C12Q 1/68

(52) U.S. Cl. 435/6; 435/5

(57) ABSTRACT

The invention provides methods for identifying markers of microbial infection, diagnosing microbial infections, and identifying the specific pathogen(s) involved in a microbial infection based on the signature patterns of gene expression induced in a host in response to different pathogens. The invention includes compositions and kits comprising newly identified markers of infection.

### RELATED APPLICATION

This application claims the benefit of U.S. Provisional Application No. 60/685,888, filed Jun. 1, 2005, which is incorporated by reference in its entirety herein

### GOVERNMENT INTEREST

The invention was made in part with Government support under a Grant awarded by the Department of Defense and by the National Institutes of Health. The Government may have certain rights to the invention.

### FIELD OF THE INVENTION

The present invention relates to methods for diagnosis, prognosis, and treatment of microbial infections. More specifically, the invention relates to methods for identifying pathogens and markers of infection based on the signature patterns of gene expression induced in a host in response to different pathogens. The invention includes compositions and kits comprising markers of infection by influenza A virus and/or S. pneumoniae

### BACKGROUND

Respiratory tract infections are the most common cause of human illness and account for the majority of lost workdays and school absences each year.¹ Most upper respiratory tract infections are caused by viruses or bacteria, and range in severity from the self-limited common cold to life-threatening avian influenza and pneumonia. Influenza viruses, the causative agent for influenza (a highly contagious respiratory viral illness), can be classified into three distinct types (A, B and C), each of which exhibits high antigenic diversity. Influenza A virus triggers frequent (typically annual) and serious epidemics, resulting in considerable morbidity and mortality, and occasional pandemics involving millions of people worldwide. Outbreaks of influenza B and C occur less frequently, and generally involve mild or asymptotic illness.
¹ According to a recent report by the Center for Disease Control (CDC), an estimated 10-20% of Americans suffer from influenza each year, resulting in an average of over 100,000 hospitalizations and 30,000 deaths from flu-related complications (http://www.dcd.gov/flu/keyfacts.htm).

Several global pandemics have occurred during the last century, most notably the “Spanish flu” of 1918-1919, which killed an estimated 20-40 million people worldwide (see, e.g., Stephenson, I., and K. G. Nicholson, Eur. Respir. J. (2001) 17:1282-1293). Complications of acute influenza include viral and secondary bacterial pneumonias, exacerbations of pre-existing cardiopulmonary disease in the elderly, and a variety of respiratory illnesses in adults and children. Influenza A infection has been implicated, for example, in the development of acute otitis media (AOM), a serious childhood illness typically involving carriers of Streptococcus pneumoniae (S. pneumoniae). It is believed that influenza A increases accessibility of host receptors for S. pneumoniae, thereby facilitating a dual viral/bacterial infection, which reportedly produces a synergistic effect (see Tong, et al., Microbial Pathogenesis (2004) 37:193-204).

Many experts believe the combination of rapid global population growth and increased frequency of intercontinental travel are contributing factors in both the reemergence of known pathogens and surfacing of novel pathogens and diseases, such as the 2003 epidemic of severe acute respiratory syndrome (SARS) coronavirus and the recent outbreaks of the highly pathogenic avian influenza virus A (H5N1) in Asia and Europe (see, e.g., Trampuz et al., Mayo Clin. Proc. (2004) 79(4):523-530). The influenza H5N1 strain has killed more than 1,000 migratory birds in China, and at least 130 laboratory-confirmed human cases and 67 deaths in Asia since December 2003.² Some experts believe that birds, which can act as carriers of influenza, may spread highly dangerous strains such as H5N1 to other species, including humans. The recent announcement that at least 500 wild birds across five different species have died from this highly infectious virus supports this concern. Many experts now suspect that the virus's genes may have mutated or reassorted, and may potentially acquire human-to-human transmissibility, thus sparking fears of an impending global pandemic.
² See World Health Organization (WHO) report of Nov. 17, 2005 at www.who.int/csr/disease/avain_influenza/country/cases_table_{—}2005_{—}11_{—}17/en/index.htm.

Clinical manifestations of upper respiratory tract infections generally include sore throat, runny nose, fever, cough, headache, and general malaise. The fact that the vast majority of respiratory infections, both viral and bacterial, produce similar flu-like symptoms makes the diagnosis of a specific infection (influenza, pneumonia or other respiratory illness) based solely on clinical symptoms virtually impossible. To further confound the problem, respiratory pathogens in general, and influenza in particular, show high antigenic diversity. Thus, each of the various classes of respiratory pathogens comprises numerous strains, subtypes and/or serotypes, which can take days to identify using conventional diagnostic techniques. Early diagnosis, which has always been important for therapeutic and prognostic reasons, has become a global health crisis given the growing risks of pandemic strains emerging. Early detection and identification of highly contagious and virulent strains, or of even a single case of human-to-human transmission of an avian influenza virus, at an early stage when containment might be feasible are critical steps in averting a potentially devastating pandemic.

Current diagnostic techniques, particularly those involving cell culture and serologic testing, can take days or even weeks to complete, which may be too late to effectively treat or contain a highly contagious infection. A need therefore exists for an accurate and rapid means to detect respiratory infections and identify the specific causative agent.

### SUMMARY OF THE INVENTION

The present invention is based, at least in part, on the discovery that the expression of certain genetic markers is altered in mammalian subjects in response to infection by a microorganism as compared to normal subjects, and that the expression patterns provide a based for distinguishing pathogens and diagnosing specific infections. Since different pathogens have distinct signature profiles, the differentially expressed genes can be used as markers of infection. The present invention provides methods, biochips (e.g., cDNA microarrays), and kits for diagnosing, prognosing, and monitoring the course of infection based on the aberrant expression of these genetic markers, as well as providing information useful in the selection of appropriate treatment regimes, drug screening, and the development and optimization of vaccines.

In one aspect, the invention relates to a method of assessing whether a subject is infected with an influenza A virus or Streptococcus pneumoniae (S. pneumoniae). The method comprises comparing (a) the level of expression of an influenza A infection marker or a S. pneumoniae infection marker in a subject sample; and (b) the normal level of expression of the influenza A infection marker or the S. pneumonia infection marker in a control sample. The influenza A infection marker may be selected from the group of markers listed in Tables 2 or 3, and the S. pneumoniae infection marker may be selected from the group of markers listed in Tables 4 or 5. A detectable difference in the level of expression of the influenza A or S. pneumoniae infection marker in the subject sample and the normal level of expression of the influenza A or S. pneumoniae infection marker is an indication that the subject is infected with influenza A virus or S. pneumonaie. The influenza A or S. pneumoniae infection marker may be a host immune response gene, and the subject sample may be obtained from the blood (e.g., whole blood, plasma, serum, or a nucleic acid molecule isolated or prepared from the blood), lymphoid tissue, spleen or lung of the subject. The level of expression of the influenza A or S. pneumoniae infection marker may be determined by any means known in the art, such as by microarray analysis or real-time quantitative RT-PCR. In certain embodiments, the method involves hybridization of a nucleic acid molecule isolated or prepared from the subject sample and the influenza A or S. pneumoniae infection marker. The nucleic acid molecule or the influenza A or S. pneumoniae infection marker may be a complementary DNA (cDNA) molecule, for example a cDNA molecule between 15 and 40 nucleotides in length. The level of expression of the influenza A or S. pneumoniae infection marker in the subject sample may be significantly different from the level of expression in the control sample.

The present invention is also based, at least in part, on the discovery that the diagnosis of infection, for example with influenza A virus or S. pneumoniae, can be made based on sequence similarities between marker genes of different species. The method involves comparing the level of expression of an influenza A infection marker or a S. pneumoniae infection marker in a subject sample with the normal level of expression of the influenza A infection marker or the S. pneumoniae infection marker in a control sample. The influenza A infection marker may have a nucleotide sequence corresponding to a mouse influenza marker gene listed in Tables 2 or 3, and the S. pneumoniae infection marker may have a nucleotide sequence corresponding to a mouse S. pneumoniae marker gene listed in Tables 4 or 5. A detectable difference in the level of expression of the influenza A or S. pneumoniae infection marker in the subject sample and the normal level of expression in the control sample is an indication that the subject is infected with influenza A virus or S. pneumoniae. The influenza A and S. pneumoniae infection markers may be human immune response genes. The level of expression of the influenza A or S. pneumoniaeio infection marker may be determined using any of a variety of known techniques, for example using microarray analysis or real-time quantitative RT-PCR as described herein.

The invention also relates to a method of assessing whether a subject is infected with both influenza A virus and S. pneumoniae. The method involves making a comparison between the level of expression of an influenza A and an S. pneumoniae infection marker in a subject sample and the normal level of expression in a control sample. The influenza A and the S. pneumoniae infection marker may correspond to the mouse markers listed in Table 6. A detectable difference in the level of expression of the influenza A and S. pneumoniae infection marker in the subject sample and the control sample is indicative of infection with influenza A virus and S. pneumonaie.

In another aspect, the invention relates to a method of identifying markers of infection by influenza A virus or S. pneumoni. The method comprises the steps of: (a) obtaining a sample from a subject that is infected with an influenza A virus or S. pneumonia; (b) isolating and labeling mRNA from the sample; (c) detecting labeled mRNA from the sample to produce a gene expression profile; and (d) comparing the gene expression profile from step (c) with a reference gene expression profile of a control to determine a difference in the level of expression of at least one gene. A difference in level of expression may be determined by microarray analysis or real-time quantitative RT-PCR, wherein the difference in the level of expression of at least one gene identifies the gene(s) as a marker of infection by influenza A virus or S. pneumonia. The influenza A or S. pneumoniae infection marker may be a host immune response gene, and the detection step may be performed using microarray analysis or real-time quantitative RT-PCR. The subject may be a human or non-human animal (e.g., a mouse), and the gene(s) may correspond to a marker gene selected from at least one of Tables 2-6.

The invention also relates to a method of assessing whether a subject (e.g., human) is infected with influenza A virus or S. pneumoniae by comparing the levels of expression of a subject (e.g., human) influenza A or an S. pneumoniae infection marker with the normal level of expression in a control sample. The influenza A or S. pneumoniae infection marker may be a human influenza gene having a nucleotide sequence corresponding to a mouse influenza response gene listed in Tables 2 or 3, and wherein the S. pneumoniae infection marker may be a human pneumoniae response gene having a sequence corresponding to a mouse pnueomiae response gene listed in Tables 4 or 5. A detectable difference in the level of expression of the influenza A or S. pneumoniae infection marker in the subject sample and the normal level of expression of the influenza A or S. pneumoniae infection marker in the control sample is an indication that the subject is infected with influenza A virus or S. pneumoniae.

In another aspect, the invention relates to a gene expression profile comprising the group of genes or a group of human genes corresponding to the group of genes listed in one of Tables 2-6.

In still another aspect, the invention relates to a kit for diagnosing an influenza A and/or a S. pneumoniae infection in a subject. The kit may comprise a plurality of nucleic acid molecules corresponding to the group of influenza A infection marker genes listed in Tables 2 or 3; a plurality of nucleic acid molecules corresponding to the group of S. pneumoniae infection marker genes listed in Table 4 or 5; or a plurality of nucleic acid molecules corresponding to the group of influenza A and S. pneumoniae infection marker genes listed in Table 6. The nucleic acid molecules may hybridize to or have sequences corresponding to marker genes listed in one of Tables 2-6.

### BRIEF DESCRIPTION OF THE DRAWINGS

FIG. 1 provides a graphical representation of the number of genes up-regulated and down-regulated in mice at days 1 and 7 following exposure to influenza Virus A or Streptococcus pneumoniae.

FIG. 2 shows the distribution and relative numbers of genes, categorized by function, that are differentially expressed in mice in response to influenza virus A and Streptococcus pneumoniae.

FIG. 3 shows the relative changes in expression levels of 28 mouse genes at day 1 following exposure to influenza virus A/PR/8/34 and Streptococcus pneumonia.

### DETAILED DESCRIPTION OF THE INVENTION

The present invention provides methods for identifying markers of microbial infection and detecting specific disease agents based on the signature patterns of gene expression profiles induced in a mammalian host in response to different pathogens. The invention includes compositions and kits comprising markers of infection by influenza A virus and/or S. pneumoniae. The present invention is useful in the diagnosis, prophylaxis, prognosis and treatment of disease.

Definitions

As used in the context of the present invention, and unless otherwise indicated, each of the following terms shall have the meaning set forth in this section.

The articles “a” and “an” are used herein to refer to one or to more than one (i.e. to at least one) of the grammatical object of the article. By way of example, “a marker gene” means one marker gene or more than one marker gene.

“Nucleic acids” and “nucleic acid molecules” are used interchangeably to include, without limitation, single- and double-stranded nucleic acids. As used herein, the term “nucleic acid(s)” also includes DNAs or RNAs that contain one or more modified bases. Thus, DNAs or RNAs with backbones modified for stability or for other reasons are “nucleic acids.” The term “nucleic acids” as it is used herein embraces such chemically, enzymatically or metabolically modified forms of nucleic acids, as well as the chemical forms of DNA and RNA characteristic of viruses, bacteria and other cells, including for example, simple and complex cells. A nucleic acid useful in the practice of the present invention may be an isolated or purified nucleic acid or it may be an amplified nucleic acid in an amplification reaction.

When used herein in reference to a nucleic acid molecule, the terms “isolated” and “purified” mean that a naturally occurring nucleic acid molecule has been removed from its normal cellular (e.g., chromosomal) environment or is synthesized in a non-natural environment (e.g., artificially synthesized). Thus, an “isolated” or “purified” nucleotide may be in a cell-free solution or placed in a different cellular environment. The term “purified” does not imply that the sequence is the only nucleotide present, but that it is essentially free (about 90-95%, up to 99-100% pure) of non-nucleotide or nucleic acid material naturally associated with it, and thus is distinguished from isolated chromosomes.

A “marker” or “marker gene” refers to a gene that is specifically regulated in response to a microorganism or combination of microorganisms that elicit an immune response. “Marker” and “marker genes” can be used interchangeably. As used herein, a marker gene refers to a gene having a level of expression in a host in response to exposure to a pathogen that is detectably different from the level of expression in a control (i.e., a mammal that has not been exposed to the pathogen) at a corresponding time point or points. The terms “detectably different” or a “detectable difference” refer to a quantitative or qualitative difference in gene expression as determined by any one or more of a variety of methods known in the art for polynucleotide detection (e.g., electrophoresis or DNA microarray). For example, a detectable difference exists if the quantitative difference of a gene expression (i.e., increase or decrease) between two samples is about 20%, about 30%, about 50%, about 70%, about 90% to about 100% (about two-fold) or more, up to and including about 1.2 fold, 2.5 fold, 5-fold, 10-fold, 20-fold, 50-fold or more. A gene whose expression levels are detectably different between two or more samples is considered to be differentially expressed between the samples.

A marker gene may encode several different RNA transcripts, resulting from alternative splicing, processing or transcription initiation. A “marker nucleic acid” is a nucleic acid (e.g., RNA, DNA) comprising or corresponding to (in case of cDNA) the complete or a partial sequence of a RNA transcript encoded by a marker gene, or the complement of such complete or partial sequence. A “marker protein” is a protein encoded by or corresponding to a marker of the invention. The terms “protein” and “polypeptide” are used interchangeably.

An “over-expression” or “significantly higher level of expression” of a marker refers to an expression level in a sample obtained from an infected host (i.e., a mammal that has been exposed to a particular pathogen) that is greater than the standard error of the assay employed to assess expression, and may be, for example, at least twice, or three, four, five or ten times the expression level of the marker in a control sample (e.g., a sample from a healthy subject or control animal that is not infected with the pathogen). A “significantly lower level of expression” of a marker refers to an expression level in a sample that is at least twice, or three, four, five or ten times lower than the expression level of the marker in a control sample. As used herein, the term “significantly different” means the level of expression of a marker in a host sample is significantly higher or lower than the level of expression in the control sample. The terms “normal” and “normal level of expression” refer to the level of expression of a marker gene in a sample from a healthy (uninfected) subject or control animal.

“Complementary” refers to the broad concept of sequence complementarity between regions of two nucleic acid strands or between two regions of the same nucleic acid strand. For example, an adenine residue of a first nucleic acid region is capable of forming specific hydrogen bonds (“base pairing”) with a residue of a second nucleic acid region which is antiparallel to the first region if the residue is thymine or uracil. Similarly, a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. The first region may comprise a first portion and the second region may comprise a second portion, whereby, when the first and second portions are arranged in an antiparallel fashion, at least about 50%, at least about 75%, at least about 90%, or at least about 95% of the nucleotide residues of the first portion are capable of base pairing with nucleotide residues in the second portion. In some instances, all nucleotide residues of the first portion may be capable of base pairing with nucleotide residues in the second portion.

A “cDNA,” also called a complementary-DNA, refers to a product of a reverse transcription reaction from an mRNA transcript that is complementary to the mRNA template. “RT-PCR” refers to reverse transcription polymerase chain reaction and results in production of cDNAs that encode the sequence of the mRNA template(s).

The term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue-specific manner.

The term “probe” refers to any molecule which is capable of selectively binding to a specifically intended target molecule, for example, a nucleotide transcript or protein encoded by or corresponding to a marker. Probes can be either synthesized by one skilled in the art, or derived from appropriate biological preparations. For purposes of detection of the target molecule, probes may be specifically designed to be labeled, as described herein. Examples of molecules that can be utilized as probes include, but are not limited to, RNA, DNA, proteins, antibodies, and organic molecules.

The terms “microbe” and “pathogen” may be used interchangeably herein and refer to any one of a variety of infectious microorganisms including, but are not limited to, bacterial, viral, protozoal, or fungal infectious agents. Two microbes are considered distinct if they belong to different classes or types of microorganisms, different subtypes or species within the same type, or different strains within the same subtype or species. Common infectious bacteria include, but are not limited to, Escherichia coli, Salmonella, Shigella, Klebsiella, Pseudomonas, Listeria monocytogenes, Mycobacterium tuberculosis, Mycobacterium avium-intracellulare, Yersinia, Francisella, Pasteurella, Brucella, Clostridia, Bordetella pertussis, Bacteroides, Staphylococcus aureus, Streptococcus pneumonia, B-Hemolytic strep., Corynebacteria, Legionella, Mycoplasm, Ureaplasma, Chlamydia, Neisseria gonorrhea, Neisseria meningitides, Hemophilus influenza, Enterococcus faecalis, Proteus vulgaris, Proteus mirabilis, Helicobacter pylori, Treponema palladium, Borrelia burgdorferi, Borrelia recurrentis, Rickettsial pathogens, Nocardia, and Acitnomycetes. Infectious respiratory bacteria include, but are not limited to, Streptococcus pneumoniae, Haemophilus influenzae, Staphylococcus aureus, klebsiella, or legionella. Common infectious viruses include, but are not limited to, influenza viruses, human immunodeficiency virus, human T-cell lymphocytotrophic virus, hepatitis viruses, Epstein-Barr Virus, cytomegalovirus, human papillomaviruses, orthomyxo viruses, paramyxo viruses, adenoviruses, corona viruses, rhabdo viruses, polio viruses, toga viruses, bunya viruses, arena viruses, rubella viruses, and reo viruses. Infectious respiratory viruses include, but are not limited to, influenza virus type A, influenza virus type B, influenza virus type C, parainfluenza virus type 1, parainfluenza virus type 2, parainfluenza virus type 3, rhinoviruses, respiratory syncytial virus, a respiratory coronavirus, or a respiratory adenovirus. Common infectious fungi include, but are not limited to, Cryptococcus neaformans, Blastomyces dermatitidis, Histoplasma capsulatum, Coccidioides immitis, Paracoccicioides brasiliensis, Candida albicans, Aspergillus fumigautus, Phycomycetes (Rhizopus), Sporothrix schenckii, Chromomycosis, and Maduromycosis. Infectious respiratory fungi include, but are not limited to, Coccidiodes immitus, Histoplasma capsulatum or Cryptococcus neoformans.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. The first region may comprise a first portion and the second region may comprise a second portion, whereby at least about 50%, at least about 75%, at least about 90%, or at least about 95% of the nucleotide residue positions of each of the portions are occupied by the same nucleotide residue. All nucleotide residue positions of each of the portions may be occupied by the same nucleotide residue.

The term “corresponding sequence” refers to a nucleotide sequence that is similar or homologous to another nucleotide sequence, whether the exact position is identical or different from the molecule to which the similarity or homology is measured. The term “corresponding to” refers to the sequence similarity, and not the numbering of the amino acid residues or nucleotide bases. Thus, the term “corresponding human sequence” refers to a human sequence that is similar to or shares sequence homology with a non-human sequence.

“Proteins of the invention” encompass marker proteins and their fragments; variant marker proteins and their fragments; peptides and polypeptides comprising an at least 15 amino acid segment of a marker or variant marker protein; and fusion proteins comprising a marker or variant marker protein, or an at least 15 amino acid segment of a marker or variant marker protein.

The terms “individual” and “subject” refer to a mammal, either human or non-human, that (1) has, may have, or is suspected to have a microbial infection; (2) has, may have, or is suspected to have been exposed to a pathogen; and/or (3) is not known to have a microbial infection and/or to have been exposed to a pathogen. Although the examples described herein involve mouse models of microbial infection, the methods of the invention are generally applicable to any mammal that is capable of mounting an immune reaction in response to exposure to a pathogen. As used herein, and unless otherwise clear from the context, the terms “individual” and “subject” refer to a human or animal that has not been intentionally exposed to a pathogen (e.g., for the purpose of inducing an immune response). The terms “host” and “animal host,” unless otherwise clear from the context, refer to a mammal that either is or may become infected by a pathogen, either through exposure to an infected mammal or by direct (unintentional or intentional) exposure to a pathogen (e.g., for the purpose of inducing an immune response). Also as used herein, and depending upon the context, the term “host” refers to an organism (mammal) susceptible to infection by a pathogen. Thus, the term “host gene” means a mammalian gene, as compared to a gene from a microorganism (“microbial gene”). The host gene may be a gene involved in immunity, for example a mammalian gene that is regulated in response to infection by a specific pathogen. A “control,” as used herein, refers to a healthy human or animal that is not infected with a particular pathogen. The methods of the invention are useful in the fields of medicine, veterinary medicine, animal sciences and the like.

As used herein, the terms “sample” and “subject sample” refer to a biological material which is isolated from its natural environment (i.e., derived or obtained from a mammalian subject) and contains a nucleic acid molecule or polypeptide. A “sample” according to the invention may consist of purified or isolated nucleic acid and/or a polypeptide, or it may comprise a biological sample such as a tissue sample, a biological fluid sample, or a cell sample comprising a nucleic acid and/or polypeptide. A biological fluid includes blood, plasma, sputum, urine, cerebrospinal fluid, lavages, and leukophoresis samples.

The presence of a gene expression product (e.g., cDNA, RNA or EST) may be “detected” by any method known to those of skill in the art or taught in numerous texts and laboratory manuals (see for example, Ausubel, et al., “Short Protocols in Molecular Biology,” 3rd ed. (John Wiley & Sons, Inc.; 1995). Exemplary methods of detection include, but are not limited to, microarray, RNA fingerprinting, Northern blotting, polymerase chain reaction, real-time quantitative RT-PCR, differential display, ligase chain reaction, Qbeta replicase, isothermal amplification method, strand displacement amplification, transcription based amplification systems, nuclease protection (SI nuclease or RNAse protection assays), and affinity chromatography.

The term “plurality” refers to more than one. Plurality, according to the invention, can be 2 or more, 5 or more, 10 or more, 20 or more, 50 or more, 100 or more, or 1000 or more, for example, up to the number of cDNAs corresponding to all mRNAs in a sample.

A nucleic acid molecule “hybridizes” to another nucleic acid molecule when the hybridization occurs under “stringent conditions” as described herein or as understood by those of skill in the art (see, e.g., Sambrook et al., “Molecular Cloning: A Laboratory Manual,” (New York: Cold Spring Harbor Press, 1989). “Stringent conditions” typically include (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

A “primer” refers to an oligonucleotide capable of annealing to a complementary nucleic acid template and providing a 3′ end to produce an extension product which is complementary to the nucleic acid template. The conditions for initiation and extension usually include the presence of four different deoxyribonucleoside triphosphates and a polymerization-inducing agent such as DNA polymerase or reverse transcriptase, in a suitable buffer (“buffer” includes substituents which are cofactors, or which affect pH, ionic strength, etc.) and at a suitable temperature. Primers may be single- or double-stranded, and preferably single-stranded for maximum efficiency in amplification. “Primers” useful in the present invention are less than or equal to 50 nucleotides in length, e.g., less than or equal to 50, 40, 30, 20, 15, or 10 nucleotides in length. A “probe” refers to a nucleic acid molecule having the above characteristics, except that the probe does not have to provide a 3′ end for extension. Primers and probes as described above may be also generally referred to as “oligonucleotides.”

The term “label” or “detectable label” refers to any atom or molecule which can be used to provide a detectable (e.g., quantifiable) signal, and which can be operatively linked to a nucleic acid. Labels may provide signals detectable by fluorescence, radioactivity, colorimetry, gravimetry, X-ray diffraction or absorption, magnetism, enzymatic activity, mass spectrometry, binding affinity, hybridization radiofrequency, nanocrystals and the like. A primer or probe of the present invention may be labeled so that the amplification reaction product may be “detected” by “detecting” the detectable label. See for example, DeLuca, Immunofluorescence Analysis, in “Antibody As a Tool” (Marchalonis, et al., eds.; John Wiley & Sons, Ltd.; 1982), which is incorporated by reference in its entirety herein.

A molecule is “fixed” or “affixed” to a substrate if it is covalently or non-covalently associated with the substrate such the substrate can be rinsed with a fluid (e.g. standard saline citrate, pH 7.4) without a substantial fraction of the molecule dissociating from the substrate.

A kit is any manufacture (e.g. a package or container) comprising at least one reagent, e.g. a probe, for specifically detecting the expression of at least one marker of the invention. The kit may be promoted, distributed, or sold as a unit for performing the methods of the present invention.

Description

The present invention is based, in part, on newly identified gene markers that are over- or under-expressed (i.e., differentially expressed) in a host in response to exposure to a microbe (e.g., influenza A virus and/or S. pneumoniae) as compared to the level of expression in a healthy human or control animal. Differential expression of at least one gene marker in a sample taken from a subject indicates the subject is infected with the microbe. The invention provides new markers, compositions, microarrays, kits, and methods for detecting and diagnosing a microbial infection, determining the identify of a pathogen, screening for anti-microbial drugs and treating individuals diagnosed as having a microbial infection. The compositions and methods of the invention may be applied to the detection and differentiation of any of a variety of microbial infections, to the development of new markers, and to the use of such markers to screen for drugs to treat infection, and is not intended to be limited to the exemplified pathogens or infectious respiratory agents described herein.

Early identification of pathogens is a critical first step in providing effective treatment of microbial infections, as prognosis and therapeutic outcome often depend upon the stage of infection and timing of pathogen-specific treatment regimen. In one embodiment, the present invention provides a method for identifying a pathogen that has infected a host organism (e.g., a mammal). The method comprises the steps of exposing a host to a pathogen, obtaining a sample from the host, isolating total mRNA from the sample, obtaining a gene expression profile of at least one host gene, and comparing the gene expression profile of the host gene(s) with that of a sample obtained from a healthy individual or control animal that has not been exposed to the microbe. Alternatively, rather than exposing a host to a pathogen (i.e., to induce an immune response), the host may be known to be infected with the pathogen. The gene expression profiles may be determined by any means known in the art, such as by microarray analysis containing a plurality of probes comprising host genes, for example at least one thousand, five thousand, ten thousand, or 15 thousand probes comprising host genes as exemplified herein. As discussed above, a detectable difference in the expression profiles for the gene(s) identifies the gene(s) as a marker of infection by the pathogen. Methods of isolating RNA are described herein and well known to those of skill in the art. “Gene profile” or “gene expression profile” as used herein are defined as the level or amount of transciption of a particular gene or genes as assessed by methods described herein or other methods known in the art. The gene expression profile can comprise data on one or more genes and can be measured at a single time point or over a period of time. The profile of a marker gene can be positive or negative. As used herein, “positive” means an increase in the level or amount of transcription of a particular host gene(s) relative to the level or amount of the transcript in a healthy individual or control, whereas “negative” means a decrease in the gene expression (transcription) relative to a healthy individual or control.

Pathogens can be identified by comparing the gene expression profile of a sample obtained from an subject (subject sample) with a gene expression profile obtained from a mammal of similar age, gender and species that is known to be infected with a specific pathogen. The pathogen may be a family or class of pathogens associated with a particular gene profile. For example, the pathogen can be a member of the influenza virus family (e.g., influenza A virus), a member of the Streptococcus family (e.g., Streptococcus pneumonia), or a member of the coronovirus family (e.g., SARS-CoV). Knowing the identity of the pathogen responsible for disease facilitates selection of an appropriate treatment regime, and hence prognosis and treatment outcome. Such knowledge is also useful for quarantine purposes to help contain the spread of infection, as well as for producing and optimizing vaccines for protection against infection by the pathogen.

In another aspect, the present invention relates to a method for identifying a signature pattern of gene expression in a host in response to a specific pathogen, which may then be used to diagnose infection by the pathogen. The method comprises the steps of exposing a host to a specific pathogen, obtaining a post-exposure sample from the host, isolating and labeling total mRNA from the host sample, detecting labeled mRNA to generate a host gene expression profile, and comparing the host gene expression profile (i.e., reference profile) to a gene expression profile obtained from a control sample. The differences in gene expression profiles between the host and control samples (i.e., the pattern of differentially expressed genes) represents the signature pattern of infection by the pathogen. The signature pattern of infection can then be used as a reference expression profile to which expression profiles from subject samples can be compared. Similarities between the reference expression profile and the expression profile of the subject sample can be used as a basis for diagnosing infection. or differences The presence of at least one gene that is differentially expressed A pattern of differentially expressed A signature pattern of expression profile of at least one marker gene in the gene expression profile in indicative of infection by the pathogen.

As discussed above, the present invention can be used to identify target genes associated with a host response to a specific pathogen to screen for or determine an appropriate therapeutic regimen (i.e., treatment of a patient with a pharmacological agent). The effectiveness of a therapeutic regimen can be determined by monitoring clinical manifestation of the infection in the patient undergoing treatment. Methods for determining the effectiveness of a therapeutic regimen are known in the art.

Quantitation of gene profiles from the hybridization of labeled mRNA/DNA microarray may be performed using any of a variety of methods known in the art, such as by scanning the microarrays to measure the amount of hybridization at each position on the microarray using, for example, a ScanArray™ Express Laser Scanner (PerkinElmer, Boston, Mass.). Alternatively, labeled RNA can be hybridized to a filter or other solid support containing target nucleic acids having the marker genes of the present invention. Hybridization conditions should be stringent enough to ensure specific binding between labeled RNA and target genes. Stringent hybridization conditions are known to those of ordinary skill in the art (see Sambrook et al., Molecular Cloning: A Laboratory Manual, Cold Spring Harbor Laboratory Press; Ausubel, F. M., et al., Current Protocols in Molecular Biology, Greene Publishing Assoc. and Wiley-Interscience 1987, & Supp. 49, 2000). Quantitation of specific hybridization can be performed by any suitable method including scintilation and densitometry.

In certain aspects, the present invention relates to compositions and kits comprising marker genes of influenza A and/or bacterial pneumonia. In this regard, the present invention provides a list of genes differentially expressed (i.e., regulated) in response to infection by influenza A virus, S. pneumoniae, and infections involving both pathogens (see tables 2-6). The compositions and kits comprising the marker genes or genes corresponding to the marker genes are useful for diagnosis, drug screening, prophylaxis, prognosis, and therapy of microbial infections.

The methods of the present invention may be performed using conventional molecular biology, microbiology, and recombinant DNA techniques within the skill of the art. Such techniques are explained fully in the literature. See, e.g., Sambrook, Fritsch & Maniatis, “Molecular Cloning: A Laboratory Manual (1982); “DNA Cloning: A Practical Approach,” Volumes I and II (D. N. Glover ed. 1985); “Oligonucleotide Synthesis” (M. J. Gait ed. 1984); “Nucleic Acid Hybridization” [B. D. Hames & S. J. Higgins eds. (1985)]; “Transcription and Translation” [B. D. Hames & S. J. Higgins eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984), each of which is incorporated by reference in its entirety herein.

Various aspects of the invention are described in further detail in the following subsections.

1. Mammalian Hosts

The methods of the invention are generally applicable to any organism capable of mounting an immune reaction in response to a pathogen. Thus, although any of a variety of animal models may be used to identify pathogens and markers of disease, the inventive methods are exemplified using mice, given their relatively low cost and ready availability.³ Moreover, due to similarities in their immune systems and overall genetic makeup (i.e., over 90% DNA compatibility), mice models of microbial infection are particularly useful for identifying corresponding markers of infectious disease in humans. For example, using widely available software programs, one can readily search public and private sequence databases to identifying human gene sequences corresponding to mouse markers of infection (i.e., mouse immune response genes found to be differentially expressed in response to specific pathogens). Alternatively, human markers of infection can be identified through hybridization with mouse immune response genes (markers). The human genes can be replicated using known methodologies (e.g., amplification by standard PCR) to produce microarrays for human diagnostic purposes, i.e., to verify signature patterns of gene expression induced by in humans in response to different pathogens.
³ Mouse models of infection can be obtained, for example, from commercial sources (see, e.g., http://www.jax.org) or as described in the art (see, e.g., Bitko et al. (2005), Kong et al. (2005), Yanagihara et al. (2003), Yang and Nabel. (2004), and Tsitoura et al. (2000), all of which are incorporated by reference herein.

As described more fully in the example section below, cDNA microarrays comprising a plurality of mouse genes (i.e., 15,000) were used to determine signature patterns of gene expression in response to different pathogens. Such assays (i.e., involving host response to infection) generally provide greater sensitivity than techniques involving direct detection of pathogens. Thus, because relatively low levels of expression are detectable (i.e., measurable), the methods of the present invention enable early diagnosis of infection, and hence earlier opportunities for medical intervention and containment of disease.

2. Pathogens

As discussed above for mammalian hosts, the methods of the invention are applicable to any microorganism or combination of microorganisms capable of causing a respiratory infection. As described more fully in the example section below, the present invention is exemplified using the highly contagious influenza A virus and S. pneuomia, each of which is independently capable of causing disease. In addition, these exemplified pathogens produce a combined (and reportedly synergistic) viral-bacterial infection.⁴ Using the methods of the present invention, one can identify genetic markers for the individual and/or combined viral-bacterial respiratory infections. In certain embodiments, the invention provides markers and means for diagnosing respiratory infections caused by different influenza A strains (e.g., A/PR/8/34 and H1N1), and well as a dual infection cause by S. pneumoniae and influenza A (i.e., influenza A strain A/PR/8/34).
⁴ A mouse model of dual infection with influenza virus and s. pneumoniae is described in McCullers and Webster (2001)

Influenza Infection

As used herein, the term “influenza” refers to an acute contagious respiratory disease resulting from infection by an influenza virus, including but is not limited to, a human or avian influenza virus. The term “influenza” encompasses all known types and subtypes of influenza viruses. Human influenza viruses are classified into three types (A, B and C) based on their immunologically distinct nucleoprotein (NP) and matrix (M1) protein antigens. Influenza A (subtypes H1N1, H3N2, H5N1 and H7N7) is associated with high morbidity and mortality, has the potential to cause pandemics, and is virulet in patients of all ages (see, e.g., Trampuz et al., Mayo Clin. Proc. (2004) 79(4):523-530). Substantial genetic differences exists amongst the various subtypes of human influenza A virus, all of which are known to infect both humans and birds. Avian influenza viruses, which infect birds, encompass various subtypes, each of which comprises multiple strains of varying pathogenicity. Avian influenza H5 and H9 viruses, for example, are classified as “low pathogenic” viruses, whereas H7 is a “high pathogenic” viruses.

Bacterial Pneumonia

Bacterial pneumonia (also known as pneumococcal pneumonia) is an respiratory infection caused by Streptococcus pneumonia (S. pneumonia) that affects the small air sacs (alveoli), bronchi and surrounding tissues of the lungs. Symptoms include, inter alia, fever, difficult breathing, and chest pain. In addition to bacterial pneumonia in adults, S. pneumonia is the most common cause of otitis media, meningitis, and septicemia. S. pneumonia (a gram-positive, ovoid bacterium) produces a dense capsule that protects the pathogen from phagocytosis (Tortora, et al. 1995), and which forms the basis for serological differentiation into over 90 serotypes.

3. Identification of Infection Markers by Mouse cDNA Microarray

The expression profile of microbial infection-associated genes can be measured using, for example, microarray technology. In this method, nucleic acids of interest (e.g., cDNAs and oligonucleotides) are plated, or arrayed, on a microchip substrate. The arrayed sequences are then hybridized with specific DNA probes from a sample of interest. General methods for mRNA extraction are well known in the art and are disclosed in standard textbooks of molecular biology, including Ausubel et al., Current Protocols of Molecular Biology, John Wiley and Sons (1997). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions.

In one embodiment, cDNA microarrays used to identify the biomarkers of the invention are constructed as described in Su and Trent, 2001. Mouse cDNA clones comprising the genes of interest are initially transferred to plasmid vectors in E. coli clones, then cultured in appropriate medium for plasmid isolation. If a clone fails to grow, the microarray presumably lacks the gene. The sequence-specific primers flanking the inserted genes are used to amplify the cDNA inserts. After PCR amplification, 5 of 80 μl of PCR products are used for electrophoresis analysis to confirm the successful amplification. The remaining 75 μl of PCR products are subjected to DNA purification and then dissolved into ddH₂O to measure the concentration of DNA. Finally, purified PCR products are dissolved in 3×SSC at 200-500 ng/gl prior to printing.

Microscopy glass slides (75×25 mm) are coated with poly-L-lysine as described in Su and Trent, 2001. Prior to printing, the coated slides are individually scanned and viewed by the naked eye. Tiny “dirty spots” on the slides, particularly those within the printing area, are removed by Dust-Off (DPSXL-7105, Falcon Safety Products, Inc., Branchburg, N.J.). Only spot-free slides are used to print the cDNA clones, which in the exemplified embodiments, comprised 15,000 mouse genes, 192 controls, and various other genes for control purposes, namely 96 housekeeping genes (expression ratios), 24 Cot-1 DNAs (cross-hybridization with repetitive sequences), 24 genomic DNAs (genomic hybridization), 24 vector DNAs (plasmid contamination), and 24 non-DNA spots (background). DNAs are cross-linked to the slide by UV irradiation. UV cross-linking are carried out by placing slides on plastic wrap with array side up in a Stratagene (La Jolla, Calif.) UV 1800 Stratalinker. The slides are blocked by acylation with succinic anhydride (Aldrich, Milwaukee, Wis.), denatured in boiling ddH₂O, rinsed twice in dH₂O, air dried and stored in the dark at 25° C. After printing, slides are inspected for quality control (i.e., by hybridization with Cy3-labeled sample cDNA and Cy5-labeled control cDNA, followed by image analysis). Slide quality is assessed based on spot clarity (absence of smears), full range distribution of signal intensities (i.e., 0-65,535), signal saturation (3% minimum), and background intensities below 500 (on average) when scanned with 90-95% laser power.

Although the exemplified cDNA microarrays comprised 15,000 genes, the present invention contemplates the use of any number of genes, including, but not limited to, up to 30,000 genes or more, up to 20,000 genes, up to 15,000 genes, between 100-15,000 genes, between 500-15,000 genes, between 1,000-15,000 genes, or between 1,000-5,000 genes.

In one embodiment, gene expression is measured at days 1 and 7 following exposure to influenza virus A/PR/8/34 and/or S. pneumoniae. Of the mouse clones screened, 471 known and unknown genes were differentially expressed in at least one of the four experimental groups. 46 genes were exclusively up-regulated in mice infected with influenza virus A/PR/8/34 at day 1, but not unchanged in response to S. pneumoniae. 49 genes were exclusively up-regulated in mice infected with streptococcus pneumoniae at day 1, but unchanged in response to influenza virus A/PR/8/34. These differentially genes provide good candidate biomarkers for diagnosis of as influenza virus A and S. pneumoniae in humans.

Fewer genes were differentially expressed at day 7 post-exposure to either influenza virus A/PR/8/34 or streptococcus pneumoniae. As shown in the tables and Figures herein, gene expression profiles change rapidly in response to pathogens, and can be detected as early as 1 day after exposure. Based on differences in gene expression profiles induced by different pathogens (e.g., influenza virus A/PR/8/34 versus S. pneumoniae), microarray data provides excellent data for distinguishing between pathogens and hence diagnosing specific infections, including those that cause similar clinical symptoms.

The microarrays of the invention can contain genes that react exclusively to either S. pneumoniae or influenza virus, with the genes either marked or strategically placed to avoid confusion. Similarly, the microarray may contain genes up-regulated, down-regulated or both, for either or both infectious agents, and at one or more time points.

Additionally, the microarray may contain a set of control genes. These genes would be known to be unaffected by the mouse's (or human's) exposure to either S. pneumoniae or the influenza virus and would yield a negative result when exposed to the RNA of the “patient.”

The discovery of the up-regulation and down-regulation of specific genes in mice in response to exposure to a Streptococcus pneumoniae and influenza virus A, along with a development of a microarray comprising the up-regulated and down-regulated genes can be used to both develop a microarray for humans and for quick identification of these infections in humans.

The microarray for the identification of up-regulated and down-regulated genes in humans can be developed by two different methods. The first method is to develop the microarray for humans exactly the same way as developed for mice, with the exception that, given the volume of blood in a human being, the blood will be used to collect the mRNA after there has been exposure to the virus or the bacteria. Similarly, as it is unethical to wantonly infect a person with a known pathogen, daily blood samples could be drawn from individuals exposed to a person (usually of the same domicile) who has been diagnosed as suffering from one of the pathogenic illnesses. Similarly, patients diagnosed by titer, plating, or other means can have blood drawn everyday so that a similar profile of up-regulated and down regulated genes can be developed.

Accordingly, polypeptide sequences and nucleic acids that encode these sequences are contemplated that comprise at least 75%, 80%, 85%, 90%, 95%, 97%, 98%, 99% or more of each functional domain of some of the sequences. Such portions of the total sequence are very useful for diagnostics as well as therapeutics/prophylaxis.

The methods used to examine S. pneumoniae and influenza virus A can be used to test different strains of influenza virus A, influenza virus B, Bacillus anthracis, Yersinia pestis, Francisella tularensis, variola virus, and filoviruses for gene expression profiles using a microarray platform.

4. Confirming the Markers Identified by Microarray

The markers identified by cDNA microarray may be further confirmed by another nucleic acid quantification and/or sequencing method. The most commonly used methods known in the art for the quantification of mRNA expression in a sample include, but are not limited to, northern blotting and in situ hybridization (Parker & Bames, Methods in Molecular Biology 106:247-283 (1999)); RNAse protection assays (Hod, Biotechniques 13:852-854 (1992)); and reverse transcription polymerase chain reaction (RT-PCR) (Weis et al., Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS). Any of the method can be used to ascertain the relative amounts of mRNA in a sample obtained from an animal, in accordance with conventional techniques known to those persons of ordinary skill in the art.

In one embodiment, the confirmation is performed by real-time quantitative Reverse Transcriptase PCR (RT-PCR), which can be used to compare mRNA levels in different sample populations, in normal and tumor tissues, with or without drug treatment, to characterize patterns of gene expression, to discriminate between closely related mRNAs, and to analyze RNA structure.

RNA may be extracted as described above and reverse transcribed into cDNA. The derived cDNA can then be used as a template in the subsequent PCR reaction.

Real-time quantitative RT-PCR can be performed using commercially available equipment, such as, for example, ABI PRISM 7700.™. Sequence Detection System.™ (Perkin-Elmer-Applied Biosystems, Foster City, Calif., USA), or Lightcycler (Roche Molecular Biochemicals, Mannheim, Germany).

To minimize errors and the effect of sample-to-sample variation, real-time quantitative RT-PCR is usually performed using an internal standard. The ideal internal standard is expressed at a relatively constant level among different tissues, and is unaffected by the experimental treatment. RNAs frequently used to normalize patterns of gene expression are mRNAs for the housekeeping genes glyceraldehyde-3-phosphate-dehydrogenase (GAPDH), □-actin and □-globin.

While this invention has been particularly shown and described with references to preferred embodiments thereof, it will be understood by those skilled in the art that various changes in form and details may be made therein without departing from the scope of the invention encompassed by the appended claims. The teachings of all references cited herein are incorporated herein by reference.

### EXAMPLES

### Example 1 Materials and Methods

Microbes

The influenza virus A/Puerto Rico/8/34 (H1N1) were purchased from American Type Culture Collection (ATCC; Manassas, Va.; No. VR-95) and grown in MDCK cells. Streptococcus pneumoniae were purchased from ATCC (No. 10015) and grown in Todd-Hewitt broth (Difco Laboratories). The MLD₅₀ (MLD₅₀=median lethal dose) for S. pneumoniae is equivalent to 5×10⁵ cfu as quantitated on tryptic soy agar (Difco Laboratories) supplemented with 5% sheep blood.

Infection of Mice with Influenza Virus and S. pneumoniae

All experiments with infected mice were carried out in biosafety level 12 facilities in Animal Resource Center at the School of Medicine, University of Southern California. Mice were cared for in accordance with the guidelines of the Committee on Care and Use of Laboratory Animals (Institute of Laboratory Animal Resources, National Research Council). Female BALB/c ByJ mice (8-10 weeks old; Jackson Laboratories, Bar Harbor, Me.) were maintained in a biosafety level 2 facility. Healthy mice were randomly assigned to four groups (4 mice/group for each time point) for each pathogen and one group as a control (10 mice). All experimental procedures were done while mice were under general anesthesia with inhaled isoflurane 2.5% (Baxter Healthcare). Infectious agents were diluted in sterile PBS to 750 TCID₅₀ (median tissue culture infective dose) for influenza virus A/PR/8/34 and 1.25×10⁵ cfu for S. pneumoniae and administered intranasally in a volume of 100 μl (50 μl per nostril) to anesthetized mice held in an upright position. Mice were monitored at least twice a day for illness and mortality.

Isolation of Total RNA from Mouse Splenic Cells

Mice were sacrificed at day 1 and day 7 post infection following euthanasia through CO₂ inhalation. Spleen was removed from sacrificed mice and transferred into a small Petri dish, which contained RPMI1640. For isolation of splenic cells, medium was injected into the spleen using a 10-ml syringe to release splenic cells. Cell suspension was transferred to a 50 ml conical tube and the debris was settled out. The cell suspension was transferred to a new 50-ml conical tube. An additional 30 ml of RPMI1640 were added and the cells were pelleted at 1200 rpm for 5 min. The medium was aspirated and cell pellet was resuspended in 30 ml of new medium. Ten milliliters of Histopaque 1077 were drawn up using a 10-ml syringe and 18G needle; the needle was switched to the long sterile needle and the Histopaque 1077 was carefully injected beneath cell suspension. The cell suspension was centrifuged at 1700 rpm for 20 min. Everything except the pellet containing RBC and debris was poured off into a clean 50-ml conical tube and inverted gently. Sterile PBS was added to the supernatant to make 50 ml and the mixture was counted and centrifuged for 5 min at 1200 rpm to pellet cells. Total RNA was isolated from splenic cells by using the TRIZOL Reagent (Invitrogen, Carlsbad, Calif.) in accordance with the manufacturer's instructions. Total RNAs were further purified using RNeasy Mini Kit (QIAGEN, Valencia, Calif.) following manufacturer's protocol.

cDNA Microarrays

The cDNA microarrays used in this study were generated in-house as described previously (Su and Trent, 2001). Here is a brief description of the quality controls for the microarrays. The vector sequence-specific primers flanking the inserted genes were used to amplify 15,000 mouse cDNA inserts. PCR products were purified and dissolved in 3×SSC at 200-500 ng/μl for printing the microarrays on microscopy glass slides (75×25 mm) coated with poly-L-lysine. There were 15,000 mouse genes on each microarray. There were 192 controls on each cDNA microarray, including 96 housekeeping genes to control expression ratios, 24 Cot-1 DNAs to control for cross-hybridization with repetitive sequences, 24 genomic DNAs to control for genomic hybridization, 24 vector DNAs to control plasmid contamination, and 24 non-DNA spots to control for background. After printing, the slide quality was checked by hybridization with Cy3-labeled sample cDNA and Cy5-labeled control cDNA and then by image analysis.

Probe Preparation and Microarray Hybridization

Total RNAs isolated from uninfected or infected mice were labeled separately with Cyanine 3-dUTP (Cy3-dUTP) or Cyanine 5-dUTP (Cy5-dUTP) using MICRMAX Direct Labeling Kit (PerkinElmer Life Sciences Inc., Boston, Mass.). Unincorporated Cy3- and Cy5-linked dUTPs and salts were removed by using MICROCON YM-100 Centrifugal Filter Unit (Millipore, Boston, Mass.). The Cy5-(infected) and Cy3-(uninfected) labeled probes were mixed, and probe volume was reduced to near dryness by using a speed vacuum. The labeled probe mixtures were resuspended in 45 μl of hybridization buffer per microarray slide and let stand for 10 min at room temperature, with occasional mixing. The probe samples were heated at 90° C. for 2 minutes, cooled at 4° C. for 10 sec and centrifuged for 5 min at high speed to pellet any particulate matter. Forty microliters of probe mixture were added onto the edge of the cover slip. Microarray slides were incubated in a dark, humid chamber for 16 h at 65° C. The microarray slides were washed once for 15 minutes in 0.06×SSC plus 0.01% SDS, and once for 15 minutes in 0.06×SSC with gentle agitation on an orbital shaker at room temperature. The slides were span for 2 minutes at 3,000 g using a swinging bucket rotor in a 50 ml conical tube to dry the slides.

Data Analysis

After hybridization, fluorescent images of the microarray were acquired for both fluorescent dyes by ScanArray Express Laser Scanner (PerkinElmer). High quality microarray image data were digitized for construction of gene expression database including individual database for each microarray row data and a user-friendly master database. All databases included gene identification number, gene name, gene description, plate and array positions, sample name, time points, and expression signal intensities, mean values, standard deviations, background intensities, signal to noise ratios, array qualities, hybridization qualities and many others. Image segmentation, target detection, and ratio calibration methods were used to report the expression ratios of each gene on the slides (Su, Bittner et al. 2000). The ratio calibration was performed based on pre-selected internal control genes, of which ratios were normalized close to the value of 1.0. A 99% confidence interval was used to determine significantly up and down expressed genes (Su, Bittner et al. 2000). The complex images were converted into numerical data for statistic analysis of the expression levels. Data from the hybridization experiment were viewed as a normalized ratio (Cy5/Cy3) in which significant deviations from 1 (no change) are indicative of increased (>2.0) or decreased (<0.53) levels of gene expression in infected mice relative to normal mice. To explore the similarity and dissimilarity between samples and genes, supervised clustering algorithms were applied (Bittner et al. 2000 and Eisen et al. 1998). Briefly, genes in each microarray experiment were first filtered according to their measurement quality. After data filtration, high quality and differentially expressed genes were subjected to clustering software developed by Xueyan Bai and Yan A. Su based on the local maximal microarray cluster method (Wu et al., 2004) to visualize relationships between the genes and between the experiments.

Quantitative Real-time RT-PCR

Eight of the genes that were differentially expressed in response to infections were validated by quantitative real-time RT-PCR analysis. Fluorogenic probes and gene specific primers for Gnai2, Gle11, Chi313, Scampi, Ier5, Bop1 Cpsf2, Fen1 and the endogenous control □-globin were purchased from Applied Biosystems. The gene names, sequences for primers and probes, and predicted product sizes are described in Table 1 below. The gene specific probes containing a 5′ reporter, 6-carboxyfluorescein (FAM), and a 3′ non fluorescent Blackhole Quencher were used to detect the amplified products. The control gene probe was labeled with VIC at the 5′ end as a reporter and another dye TAMRA [N,N,N′,N′-tetramethyl-6-carboxyrhodamine]) at 3′ end as a quencher.

TABLE 1

| | | | | |
|---|---|---|---|---|
| Gene Name | | TM | | Product |
| (Primer and Probe) | Sequence | (° C.) | (G+C)% | Size (bp) |

| | | | | |
|---|---|---|---|---|
| Mouse Gnai2 | | | | |
| Primer: Gnai2-F | 5′-GGTGCTGGCTGAGGATGA-3′ | 58 | 61.1 | 120 |
| (18 mer) | | | | |
| Primer: Gnai2-R | 5′-TCCTTCTTGTTGAGGAAGAG-3′ | 58 | 45 | |
| (20 mer) | | | | |
| Probe^{a}: Gnai2-P | 5′-CATGCATGAGAGCATGAAGCTGT-3′ | 68 | 47.8 | |
| (23 mer) | | | | |
| Mouse Gle1l | | | | |
| Primer: Glel-F | 5′-TTCATCAATACCACTCAGGCA-3′ | 60 | 42.9 | 116 |
| (21-mer) | | | | |
| Primer: Gle-R | 5′-GGAGTGTACATGACTTTGTAC-3′ | 60 | 42.9 | |
| (20 mer) | | | | |
| Probe^{a}: Gle-P | 5′-CTGTCACCATTGAAGGACCATCC-3′ | 70 | 52.5 | |
| (23-mer) | | | | |
| Mouse Chi313 | | | | |
| Primer: Chi3-F | 5′-TGAGTGACCCTTCTAAGACTG-3′ | 62 | 45 | 103 |
| (21-mer) | | | | |
| Primer: Chi3-R | 5′-GGCTCCTTCATTCAGAAATGTA-3′ | 62 | 45 | |
| (22-mer) | | | | |
| Probe^{a}: Chi3-P | 5′-AGTACTGGCCCACCAGGAAAGTA-3′ | 72 | 54.5 | |
| (24-mer) | | | | |
| Mouse Scamp1 | | | | |
| Primer: Scam-F | 5′-GGTGCTGGCTGAGGATGA-3′ | 58 | 61.1 | 120 |
| (18 mer) | | | | |
| Primer: Scam-R | 5′-TCCTTCTTGTTGAGGAAGAG-3′ | 58 | 45 | |
| (20 mer) | | | | |
| Probea: Scam-P | 5′-CATGCATGAGAGCATGAAGCTGT-3′ | 68 | 47.8 | |
| (23 mer) | | | | |
| Mouse ler5 | | | | |
| Primer: Ler5-F | 5′-GGTGCTGGCTGAGGATGA-3′ | 58 | 61.1 | 120 |
| (18 mer) | | | | |
| Primer: Ler5-R | 5′-TCCTTCTTGTTGAGGAAGAG-3′ | 58 | 45 | |
| (20 mer) | | | | |
| Probea: Ler5-P | 5′-CATGCATGAGAGCATGAAGCTGT-3′ | 68 | 47.8 | |
| (23 mer) | | | | |
| Mouse Bop1 | | | | |
| Primer: Bob1-F | 5′-GGTGCTGGCTGAGGATGA-3′ | 58 | 61.1 | 120 |
| (18 mer) | | | | |
| Primer: Bob1-R | 5′-TCCTTCTTGTTGAGGAAGAG-3′ | 58 | 45 | |
| (20 mer) | | | | |
| Probe^{a}: Bob1-P | 5′-CATGCATGAGAGCATGAAGCTGT-3′ | 68 | 47.8 | |
| (23 mer) | | | | |
| Mouse Cpsf2 | | | | |
| Primer: Cpsf2-F | 5′-GGTGCTGGCTGAGGATGA-3′ | 58 | 61.1 | 120 |
| (18 mer) | | | | |
| Primer: Cpsf2-R | 5′-TCCTTCTTGTTGAGGAAGAG-3′ | 58 | 45 | |
| (20 mer) | | | | |
| Probe^{a}: Cpsf2-P | 5′-CATGCATGAGAGCATGAAGCTGT-3′ | 68 | 47.8 | |
| (23 mer) | | | | |
| Mouse Fen1 | | | | |
| Primer: Fen1-F | 5′-CCAGAGAACTGGCTCCACAA-3′ | 62 | 52.6 | 93 |
| (20-mer) | | | | |
| Primer: Fen1-R | 5′-TGGCTCGCTCCACTTCAGC-3′ | 62 | 61.1 | |
| (19-mer) | | | | |
| Probe^{a}: Fen1-P | 5′-CTTCCTGGAGCCAGAAGTACTGG-3′ | 72 | 54.5 | |
| (23-mer) | | | | |
| Mouse α-globin | | | | |
| Primer: AfG-F | 5′-CCACCACCAAGACCTACTTTC-3′ | 64 | 52.6 | 154 |
| (21-mer) | | | | |
| Primer: AfG-R | 5′-GCATGCAGGTCGCTCAGAG-3′ | 62 | 61.1 | |
| (19-mer) | | | | |
| Probe^{b}: AfG-P | 5′-AGCCACGGCTCTGCCCAGGTC-3′ | 72 | 70 | |
| (21-mer) | | | | |

^{a}Gene specific probes containing a 5′ reporter, 6-carboxyfluorescein (FAM), and a 3′ non fluorescent quencher (Blackhole) were used to detect the amplified products.
^{b}The control gene probe was labeled with VIC at the 5′ end as a reporter and another dye TAMRA [N,N,N,N-tetramethyl-6-carboxyrhodamine]) at 3′ end acts as a quencher.

RNA samples were amplified and analyzed in the 7500 Real Time PCR System (Applied Biosystems, Foster City, Calif.) with TaqMan One-Step RT-PCR master mixture (Applied Biosystems). Total RNA (0.5 μg) isolated from uninfected and infected mouse splenic cells at day 1 was used for quantitative real-time RT-PCR amplification with TaqMan chemistry (Applied Biosystems). The reaction mixture contained a total volume of 50 μl including both tested gene and control gene primer pairs at a concentration of 1 μM and tested gene and control gene probes at a concentration of 0.2 μM. The thermal cycling consisted of 48° C. for 30 min for reverse transcription, 95° C. for 10 min for denaturation, and 50 cycles of 95° C. for 15 s and 60° C. for 1 min for amplification. The ABI Prism 7500 sequence detector is capable of analyzing the emitted fluorescence of both tested gene and control gene dyes during amplification (on-line monitoring). Tested gene amplification results in a signal from the FAM emission wavelength (518 nm) and control gene amplification produce a signal from the VIC emission wavelength (552 nm). A positive RT-PCR is measured by the cycle number required to reach the cycle threshold (C_{T}). The C_{T} is defined as 10 times the standard deviation of the mean baseline emission calculated for PCR cycles 3 to 15 (Livak 2001).

### Results

Global Monitoring of Gene Expression in Mice After Infection with Influenza Virus A/PR/8/34 and S. pneumoniae

Microarray analysis was used to analyze the global gene expression profiles in splenic cells of mice infected with influenza virus A/PR/8/34 and S. pneumoniae. Spleen is the largest lymphatic system organ that contains germinal centers in which B lymphocytes proliferate, macrophages that ingest and process antigens, and regions that house several kinds of T lymphocytes. At day 1 and day 7 after infection, spleen samples were collected, pooled for each group and total RNAs were extracted from splenic cells. Total RNAs isolated from uninfected or infected mice were labeled separately with Cy3-dUTP or Cy5-dUTP and hybridized to cDNA microarray containing 15,000 mouse genes, as described in Materials and Methods. Hybridization intensity for each gene on the microarray was compared with the uninfected control samples. The relative abundance of transcripts from each gene was reflected by the ratio of ‘green’ to ‘red’ fluorescence measured at the array element representing that gene. The greater relative abundance of mRNA in mouse infected with pathogens resulted in a high ratio of green-labeled to red-labeled copies of the corresponding cDNA. Genes whose relative transcription levels increased by >2.0-fold were considered as up-regulated, and whose relative transcription levels decreased by <0.53-fold were considered as down-regulated. Genes whose relative transcription levels were between 0.53 and 2.0 were considered as no change.

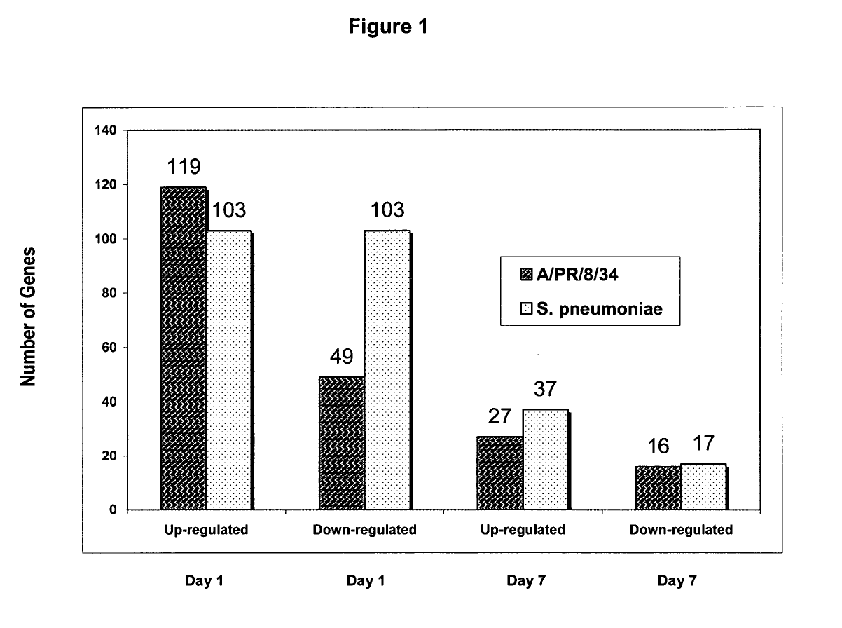

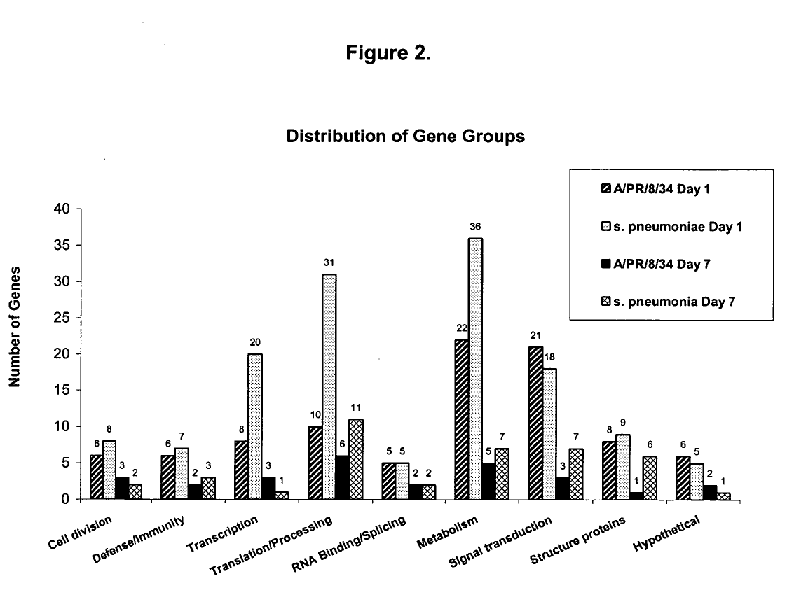

Of the 15,000 mouse clones screened, 471 known and unknown genes had a level of expression significantly different as compared with the control group (Ctr) in at least one of the four experimental groups: day 1 and day 7 after influenza virus A (FluD1 and FluD7); day 1 and day 7 after S. pneumoniae (PneD1 and PneD7). The numbers of genes, which were up-regulated or down-regulated at day 1 and at day 7 after infection with influenza virus A or S. pneumoniae, were shown in FIG. 1. There were more genes differentially expressed in mice infected with s. pneumoniae than in mice infected with influenza virus A/PR/8/34 (FIG. 1) which suggests more genes may be involved in the host defense against s. pneumoniae infection. The 471 genes showing differential expression in response to influenza virus A or S. pneumoniae were sorted into 9 groups according to their functions: cell division, defense/immunity, transcription/RNA, translation/processing, RNA binding/splicing, metabolism, signal transduction, structure proteins, hypothetical proteins and unknown. Of the 471 differentially expressed genes, 173 (36.7%) encoded unknown proteins without known functions (not shown in FIG. 2), 70 (14.9%) encoded proteins involved in metabolism, 58 (12.3%) encoded proteins related to protein translation or processing, 49 (10.4%) encoded proteins involved in signal transduction, 32 (6.8%) encoded proteins for transcription, 24 (5.1 %) encoded structure proteins, 19 (4%) encoded proteins involved in cell division and 18 (3.8%) encoded proteins related to defense and immunity, 14 (3%) encoded proteins related to RNA binding/splicing and 14 (3%) encoded hypothetical proteins (FIG. 2).

Identification of Genes Exclusively Up- or Down-regulated in Response to Influenza Virus A/PR/8/34 at Day 1

To identify genes exclusively up-regulated or down-regulated in mice infected with influenza virus A but unchanged in mice infected with s. pneumoniae at day 1, we analyzed 119 genes that were up-regulated in influenza virus A infected mice and removed genes that were also up-regulated in mice infected with S. pneumoniae. The 46 known genes exclusively up-regulated in mice infected with influenza virus A/PR/8/34 were sorted according to their fumctional groups and listed in Table 2 below.

TABLE 2

| | | | | | |
|---|---|---|---|---|---|
| | | | Gene | Locus | |
| CloneID | FluD1^{a}/Ctr^{b} | PneD1^{c}/Ctr | Name | Link^{d} | Gene Description |

Up-regulated in response to influenza virus A at day 1
Cell division

| | | | | | |
|---|---|---|---|---|---|
| H3062F01 | 2.36 | 1.84 | Dclre1a | 55947 | DNA cross-link repair 1A, PSO2 homolog (S. cerevisiae) |
| H3087H02 | 2.23 | 1.85 | Sycp3 | 20962 | synaptonemal complex protein 3 |
| H3013G04 | 2.21 | 1.44 | Mafg | 17134 | v-maf musculoaponeurotic fibrosarcoma oncogene family, |
| | | | | | protein G (avian) |

Defense/immunity

| | | | | | |
|---|---|---|---|---|---|
| H3009D08 | 6.31 | 0.92 | Chi3l3 | 12655 | chitinase 3-like 3 |
| H3124F12 | 3.46 | 1.87 | Mmp12 | 17381 | matrix metalloproteinase 12 |
| H3008A11 | 2.35 | 1.71 | Abce1 | 24015 | ATP-binding cassette, sub-family E (OABP), member 1 |
| H3022G10 | 2.08 | 1.49 | Abcf1 | 224742 | ATP-binding cassette, sub-family F (GCN20), member 1 |

Transcription

| | | | | | |
|---|---|---|---|---|---|
| H3021H11 | 8.82 | 0.94 | Fem1a | 14154 | feminization 1 homolog a (C. elegans) |
| H3031G05 | 4.67 | 1.92 | Gtf2h2 | 23894 | general transcription factor II H. polypeptide 2 |
| H3020D04 | 2.07 | 0.74 | Foxp1 | 108655 | forkhead box P1 |
| H3049G11 | 2.01 | 1.35 | Zfp282 | 101095 | zinc finger protein 282 |

Translation/processing

| | | | | | |
|---|---|---|---|---|---|
| H3099E08 | 10.99 | 1.54 | Rarsl | 109093 | arginyl-tRNA synthetase-like |
| H3011B09 | 7.69 | 1.59 | Mrpl48 | 52443 | Mitochondrial ribosomal protein L48 |
| H3141A07 | 5.42 | 0.74 | Rps4x | 20102 | ribosomal protein S4, X-linked |
| H3126H10 | 2.61 | 1.37 | Eef1a1 | 13627 | eukaryotic translation elongation factor 1 alpha 1 |
| H3113H09 | 2.24 | 1.93 | Psme3 | 19192 | proteaseome (prosome, macropain) 28 subunit, 3 |

RNA binding/splicing

| | | | | | |
|---|---|---|---|---|---|
| H3022F04 | 26.65 | 0.73 | Gle1l | 74412 | GLE1 RNA export mediator-like (yeast) |
| H3129A10 | 2.19 | 1.94 | Snrpa1 | 68981 | small nuclear ribonucleoprotein polypeptide A′ |
| H3140H02 | 2.18 | 1.68 | Cpsf2 | 51786 | cleavage and polyadenylation specific factor 2 |
| H3042H04 | 2.06 | 1.25 | Rpl27a | 26451 | ribosomal protein L27a |

Metabolism

| | | | | | |
|---|---|---|---|---|---|
| H3018F04 | 5.34 | 0.80 | Txndc4 | 76299 | thioredoxin domain containing 4 (endoplasmic reticulum) |
| H3099G04 | 5.10 | 1.00 | Fen1 | 14156 | flap structure specific endonuclease 1 |
| H3003E12 | 3.02 | 1.83 | Arl6ip2 | 56298 | ADP-ribosylation factor-like 6 interacting protein 2 |
| H3047B08 | 2.66 | 1.97 | Ctps2 | 55936 | cytidine 5′-triphosphate synthase 2 |
| H3084E05 | 2.62 | 1.17 | Gstms | 14866 | glutathione S-transferase, mu 5 |
| H3018C04 | 2.57 | 1.31 | Pld1 | 18805 | phospholipase D1 |
| H3045B10 | 2.40 | 1.56 | Asph | 65973 | aspartate-beta-hydroxylase |
| H3048E05 | 2.28 | 1.62 | Lta4h | 16993 | leukotriene A4 hydrolase |
| H3018A11 | 2.13 | 1.49 | Cyp17a1 | 13074 | cytochrome P450, family 17, subfamily a, polypeptide 1 |
| H3119F10 | 2.06 | 1.93 | Txnip | 56338 | thioredoxin interacting protein |
| H3097E03 | 2.06 | 1.37 | Cbr2 | 12409 | carbonyl reductase 2 |
| H3031F07 | 2.04 | 1.47 | Mare | 17168 | alpha globin regulatory element containing gene |

Signal transduction

| | | | | | |
|---|---|---|---|---|---|
| H3002A10 | 17.44 | 0.96 | Msl2-pending | 77853 | male-specific lethal-2 homolog (Drosophila) |
| H3002E05 | 6.01 | 0.80 | Rab1 | 19324 | RAB1, member RAS oncogene family |
| H3057E05 | 2.65 | 1.07 | Ptpn21 | 24000 | protein tyrosine phosphatase, non-receptor type 21 |
| H3108C08 | 2.28 | 1.51 | Kcne3 | 57442 | potassium voltage-gated channel, lsk-related subfamily, gene 3 |
| H3042D02 | 2.18 | 1.84 | Bag3 | 29810 | Bcl2-associated athanogene 3 |
| H3120F10 | 2.13 | 1.40 | Lcp2 | 16822 | lymphocyte cytosolic protein 2 |
| H3019D04 | 2.05 | 1.87 | Pfpl | 56093 | pore forming protein-like |
| H3108A06 | 2.05 | 1.84 | Tnfrsf19 | 29820 | tumor necrosis factor receptor superfamily, member 19 |

Structure proteins

| | | | | | |
|---|---|---|---|---|---|
| H3008B12 | 5.60 | 0.59 | Flnb | 286940 | filamin, beta |
| H3020C12 | 4.75 | 2.00 | Macf1 | 11426 | microtubule-actin crosslinking factor 1 |
| H3021B02 | 2.40 | 1.46 | Krt1-18 | 16668 | keratin complex 1, acidic, gene 18 |
| H3022G09 | 2.21 | 1.66 | Krt1-19 | 16669 | keratin complex 1, acidic, gene 19 |
| H3032B09 | 2.05 | 1.60 | Mesdc2 | 67943 | mesoderm development candiate 2 |
| H3143A06 | 2.01 | 1.60 | Tuba4 | 22145 | tubulin, alpha 4 |

Down-regulated in response to influenza virus A at day 1

| | | | | | |
|---|---|---|---|---|---|
| H3026F02 | 0.26 | 0.61 | Gpx1 | 14775 | glutathione peroxidase 1 |
| H3113G10 | 0.36 | 0.63 | Ilk | 16202 | integrin linked kinase |
| H3120G02 | 0.40 | 0.75 | Gnai2 | 14678 | guanine nucleotide binding protein, alpha inhibiting 2 |
| H3118H07 | 0.44 | 0.72 | Ccnl2 | 56036 | cyclin L2 |
| H3024D08 | 0.45 | 0.59 | Tuba2 | 22143 | tubulin, alpha 2 |
| H3023G08 | 0.46 | 0.62 | Ddx5 | 13207 | DEAD (Asp-Glu-Ala-Asp) box polypeptide 5 |
| H3131A12 | 0.46 | 0.82 | Pdcd4 | 18569 | programmed cell death 4 |
| H3081H11 | 0.52 | 0.72 | Psmd8 | 57296 | proteasome (prosome, macropain) 26S subunit, non-ATPase, 8 |
| H3131A08 | 0.52 | 0.94 | Ssr2 | 66256 | signal sequence receptor, beta |
| H3102B09 | 0.52 | 1.21 | Rad23b | 19359 | RAD23b homolog (S. cerevisiae) |

^{a}FluD1 represents day 1 after infection of mice with influenza virus A/PR/8/34.
^{b}Control represents uninfected control mice.
^{c}PneD1 represents day 1 after infection of mice with Strptococcus pneumoniae.
^{d}LocusLink is superceded by Entrez Gene. LocusLink number is the same as GeneID and can be used to search gene database at the NIH web site: http:www.ncbi.nlm.nih.gov/query.fcgi?db=gene.

We identified 11 genes whose expression increased more than 5-fold at day 1 after infection with influenza virus A/PR/8/34. These 11 genes include GLE1 RNA export mediator-like gene (Gle11, up 26.6-fold), male-specific lethal-2 homolog gene (Ms12, 17.4-fold), arginyl-tRNA synthetase-like gene (Rars1, up 11-fold), feminization 1 homolog a gene (Fem1a, up 8.8-fold), mitochondrial ribosomal protein L48 gene (Mrp148, up 7.7-fold), chitinase 3-like 3 gene (Chi313, up 6.3-fold), Rab1 gene (member of Ras oncogene family, up 6-fold), filamin beta gene (Flnb, up 5.6-fold), and flap structure specific endonuclease 1 gene (Fen1, up 5.1-fold). Chi313 gene encodes a macrophage protein that is transiently expressed during inflammation (Chang et al. 2001). Fen1 is a structure-specific endonuclease implicated for nucleotide excision repair (Harrington, et al. 1994).

The 10 known genes exclusively down-regulated (Table 2) in mice infected with influenza virus A/PR/8/34 but unchanged in mice infected with s. pneumoniae include glutathione peroxidase 1 gene (Gpx1, down 3.8-fold), integrin linked kinase gene (Ilk, down 2.7-fold), guanine nucleotide binding protein alpha inhibiting 2 (Gnai2, down 2.5-fold), cyclin L2 gene (Ccn12, down 2.2-fold), tubulin alpha 2 gene (Tuba2, down 2.2-fold), programmed cell death 4 gene (Pdcd4, down 2.1-fold) and proteasome 26S subunit 8 gene (Psmd8, down 1.92-fold).

Identification of Genes Exclusively Up- or Down-regulated in Response to Influenza Virus A/PR/8/34 at Day 7

At day 7 after infection of mice with influenza virus A/PR/8/34, the number of genes exclusively up-regulated or down-regulated is much less than that at day 1. This result indicates that the host response to infection by influenza virus A is very quick and there are many genes differentially expressed as an early response to the pathogen. We identified 12 known genes exclusively up-regulated in mice infected with influenza virus A at day 7 (see Table 3 below).

TABLE 3

| | | | | | |
|---|---|---|---|---|---|
| | FluD7^{a}/ | | | Locus | |
| CloneID | Ctr^{b} | PneD7^{c}/Ctr | Gene Name | Link^{d} | Gene Description |

Up-regulated in response to influenza virus A at day 7

| | | | | | |
|---|---|---|---|---|---|
| H3017C09 | 3.76 | 0.92 | Taf13 | 99730 | TAF13 RNA polymerase II, TATA box binding protei [symbol] |
| | | | | | (TBP)-associated factor |
| H3068G09 | 3.04 | 1.15 | Spin | 20729 | spindlin |
| H3009G06 | 2.88 | 1.00 | Papss1 | 23971 | 3′-phosphoadenosine 5′-phosphosulfate synthase 1 |
| H3107G12 | 2.82 | 0.73 | Crot | 74114 | carnitine O-octanoyltransferase |
| H3011A09 | 2.77 | 0.85 | Vps33a | 77573 | vacuolar protein sorting 33A (yeast) |
| H3121F09 | 2.49 | 1.00 | Terf1 | 21749 | telomeric repeat binding factor 1 |
| H3104B06 | 2.41 | 1.24 | Nedd4 | 17999 | neural precursor cell expressed, developmentally |
| | | | | | down-regulted gene 4 |
| H3108A03 | 2.29 | 0.96 | Apobec1 | 11810 | apolipoprotein B editing complex 1 |
| H3126F10 | 2.21 | 0.76 | Sms | 20603 | spermine synthase |
| H3025D07 | 2.20 | 1.51 | Mki67 | 17345 | antigen identified by monoclonal antibody Ki 67 |
| H3138A03 | 2.18 | 0.98 | Jak1 | 16451 | Janus kinase 1 |
| H3024C09 | 2.11 | 1.07 | Sfrs3 | 20383 | splicing factor, arginine/serine-rich 3 (SRp20) |

Down-regulated in response to influenza virus A at day 7

| | | | | | |
|---|---|---|---|---|---|
| H3146E11 | 0.35 | 0.58 | Rab7 | 19349 | RAB7, member RAS oncogene family |
| H3011C11 | 0.46 | 0.75 | Manba | 110173 | mannosidase, beta A, lysosomal |
| H3146A10 | 0.46 | 0.71 | Cfc1 | 12627 | cripto, FRL-1, cryptic family 1 |
| H3002B10 | 0.47 | 0.77 | Centg3 | 213990 | centaurin, gamma 3 |
| H3002D08 | 0.48 | 0.54 | Abcb8 | 27417 | ATP-binding cassette, sub-family B (MDR/TAP), |
| | | | | | member 8 |
| H3129F06 | 0.50 | 0.69 | Ubb | 22187 | ubiquitin B |
| H3129F04 | 0.50 | 0.53 | Atp5f1 | 11950 | ATP synthase, H+ transporting, mitochondrial F0 |
| | | | | | complex, subunit b, isoform 1 |
| H3138E05 | 0.52 | 0.68 | Mcm7 | 17220 | minichromosome maintenance deficient 7 (S. cerevisiae) |

^{a}FluD7 represents day 7 after infection of mice with influenza virus A/PR/8/34.
^{b}Control represents uninfected control mice.
^{c}PneD7 represents day 7 after infection of mice with Strptococcus pneumoniae.
^{d}LocusLink is superceded by Entrez Gene. LocusLink number is the same as GeneID and can be used to search gene database at the NIH web site: http:www.ncbi.nlm.nih.gov/query.fcgi?db=gene.

These known genes include TAF13 RNA polymerase II,TATA box binding protein gene (Taf13, up 3.76-fold), spindling gene (Spin, up 3-fold), 3′-phosphoadenosine 5′-phosphosulfate synthase 1 gene (Papss1, up 2.9 fold), carnitine O-octanoyltransferase (Crot, up 2.8 fold), vacuolar protein sorting 33A gene (Vps33a, up 2.8-fold), telomeric repeat binding factor 1 gene (Terf1, up 2.5 fold), spernine synthase gene (Sms, up 2.2 fold), Janus kinase 1 gene (Jak1, up 2.2-fold) and splicing factor gene (Sfrs3, up 2.1 fold). Jak kinases (Jak1, Jak2 and Jak3) are non-receptor tyrosine kinases which couple to cytokine receptors, and are activated by cytokines. STATs (signal-transducting activators of transcription) are the principle substrates of Jak kinases and mediators of cytokine signaling. Jakl is required for signaling from IL-2, IL-6 and interferon receptors. Jak1 may participate in the cell-mediated immune response to the influenza virus A infection at day 7 after infection. However, it will be necessary to confirm the expression level of Jakl gene before we could make any conclusions. There were 8 known genes exclusively down-regulated at day 7 after infection of mice with influenza virus A/PR/8/34 (Table 3). These genes include Ras oncogene family member Rab7 gene (Rab7, down 2.9-fold), lysosomal mannosidase beta A gene (Manba, down 2.2-fold), centaurin, gamma 3 gene (Centg3, down 2.1 fold), ATP-binding cassette sub-family B member 8 gene (Abcb8, down 2.1 fold) and ubiquitin B gene (Ubb, down 2-fold).

Identification of Genes Exclusively Up- or Down-regulated in Response to S. pneumoniae at Day 1

We identified 49 known genes exclusively up-regulated in mice infected with S. pneumoniae at day 1 but unchanged in mice infected with influenza virus A/PR/8/34 (see Table 4 below). These 49 genes were sorted according to their functional groups and listed in Table 4. We identified 8 known genes whose expression levels increased more than 5-fold after infection with S. pneumoniae. These 8 genes include folate receptor 4 gene (Folr4, up 10.6-fold), mitochondrial ribosomal protein L20 gene (Mrp120, up 7.14-fold), lysosomal H⁺ transporting ATPase gene (Atp6v1h, 7-fold), selenoprotein K gene (Selk, 6.1-fold), aryl hydrocarbon receptor nuclear translocator-like gene (Arnt1, 5.7-fold), secretory carrier membrane protein 1 gene (Scamp1, 5.6-fold) and nuclear receptor subfamily 5 group A member 2 gene (Nr5a2, 5-fold).

TABLE 4

| | | | | | |
|---|---|---|---|---|---|
| | PneD1^{a}/ | | Gene | Locus | |
| CloneID | Ctr^{b} | FluD1^{c}/Ctr | Name | Link^{d} | Gene Description |

Up-regulated in response to S. pneumoniae at day 1
Cell division

| | | | | | |
|---|---|---|---|---|---|
| H3002E09 | 2.27 | 1.91 | Sfn | 55948 | stratifin |
| H3068E12 | 2.07 | 0.83 | Cul1 | 26965 | cullin 1 |

Defense/immunity

| | | | | | |
|---|---|---|---|---|---|
| H3087G09 | 6.10 | 0.81 | Selk-pending | 80795 | selenoprotein K |

Transcription

| | | | | | |
|---|---|---|---|---|---|
| H3034A04 | 4.02 | 0.92 | Mtf2 | 17765 | metal response element binding transcription factor 2 |
| H3042G11 | 3.22 | 1.17 | Ptges2 | 96979 | prostaglandin E synthase 2 |
| H3115E12 | 2.48 | 1.86 | Bat4 | 81845 | HLA-B associated transcript 4 |
| H3049G09 | 2.10 | 1.56 | Zfp276 | 57247 | zinc finger protein (C2H2 type) 276 |
| H3056C11 | 3.33 | 1.27 | Gcipip- | 68592 | GCIP-interacting protein p29 |
| | | | pending | | |

Translation/processing

| | | | | | |
|---|---|---|---|---|---|
| H3142G09 | 7.14 | 0.79 | Mrpl20 | 66448 | mitochondrial ribosomal protein L20 |
| H3105G09 | 5.74 | 0.96 | Arntl | 11865 | aryl hydrocarbon receptor nuclear translocator-like |
| H3093F11 | 5.57 | 0.92 | Scamp1 | 107767 | secretory carrier membrane protein 1 |
| H3087G01 | 5.04 | 1.86 | Nr5a2 | 26424 | nuclear receptor subfamily 5, group A, member 2 |
| H3078G02 | 3.57 | 1.00 | Nek7 | 59125 | NIMA (never in mitosis gene a)-related expressed |
| | | | | | kinase 7 |
| H3006G12 | 2.64 | 1.00 | Rpl28 | 19943 | ribosomal protein L28 |
| H3051G01 | 2.60 | 1.22 | Rps25 | 75617 | ribosomal protein S25 |
| H3115A01 | 2.36 | 1.27 | Laptm5 | 16792 | lysosomal-associated protein transmembrane 5 |
| H3104B06 | 2.25 | 1.30 | Nedd4 | 17999 | neural precursor cell expressed, developmentally |
| | | | | | down-regulted gene 4 |
| H3024G10 | 2.20 | 1.10 | G2an | 14376 | alpha glucosidase 2, alpha neutral subunit |
| H3096G11 | 2.14 | 1.45 | Copb2 | 50797 | coatomer protein complex, subunit beta 2 (beta prime) |
| H3113C12 | 2.14 | 1.35 | Rpl3 | 27367 | ribosomal protein L3 |
| H3009C09 | 2.07 | 1.16 | Ndufaf1 | 69702 | NADH dehydrogenase (ubiquinone) 1 alpha |
| | | | | | subcomplex, assembly factor 1 |
| H3016G12 | 2.04 | 1.47 | Icmt | 57295 | isoprenylcysteine carboxyl methyltransferase |

RNA binding/splicing

| | | | | | |
|---|---|---|---|---|---|
| H3051G02 | 2.21 | 1.01 | Cpeb3 | 208922 | cytoplasmic polyadenylation element binding protein 3 |
| H3123G10 | 2.11 | 0.64 | Mgat1 | 17308 | mannoside acetylglucosaminyltransferase 1 |
| H3019E04 | 2.19 | 1.60 | Rnpc1 | 56190 | RNA-binding region (RNP1, RRM) containing 1 |

Metabolism

| | | | | | |
|---|---|---|---|---|---|
| H3054B05 | 10.64 | 0.77 | Folr4 | 64931 | folate receptor 4 (delta) |
| H3132G09 | 6.98 | 0.95 | Atp6v1h | 108664 | ATPase, H+ transporting, lysosomal 50/57 kDa, V1 |
| | | | | | subunit H |
| H3092C09 | 6.25 | 1.27 | Zdhhc6 | 66980 | zinc finger, DHHC domain containing 6 |
| H3038C09 | 4.95 | 1.37 | Prpsap2 | 212627 | phosphoribosyl pyrophosphate synthetase-associated |
| | | | | | protein 2 |
| H3069G01 | 4.70 | 1.23 | Cept1- | 99712 | choline/ethanolaminephosphotransferase 1 |
| | | | pending | | |
| H3016A04 | 3.89 | 1.05 | Slc38a1 | 105727 | solute carrier family 38, member 1 |
| H3065C01 | 3.51 | 1.74 | Atp5j2 | 57423 | ATP synthase, H+ transporting, mitochondrial F0 |
| | | | | | complex, subunit f, isoform 2 |
| H3056C09 | 3.28 | 1.08 | Gpi1 | 14751 | glucose phosphate isomerase 1 |
| H3011H07 | 3.09 | 0.70 | Oas1a | 246730 | 2′-5′ oligoadenylate synthetase 1A |
| H3073G12 | 2.80 | 1.34 | Th | 21823 | tyrosine hydroxylase |
| H3045A12 | 2.49 | 1.77 | Hba-a1 | 15122 | hemoglobin alpha, adult chain 1 |
| H3022A02 | 2.41 | 1.39 | Sepp1 | 20363 | selenoprotein P, plasma, 1 |
| H3045C12 | 2.14 | 1.35 | Acadl | 11363 | acetyl-Coenzyme A dehydrogenase, long-chain |
| H3011C11 | 2.10 | 1.66 | Manba | 110173 | mannosidase, beta A, lysosomal |

Signal transduction

| | | | | | |
|---|---|---|---|---|---|
| H3105G01 | 4.19 | 1.64 | Dtx3 | 80904 | deltex 3 homolog (Drosophila) |
| H3008A09 | 2.91 | 1.64 | Arhb | 11852 | ras homolog gene family, member AB |
| H3099A08 | 2.61 | 1.52 | Esr1 | 13982 | estrogen receptor 1 (alpha) |
| H3038C04 | 2.02 | 1.75 | Pgk1 | 18655 | phosphoglycerate kinase 1 |

Structure proteins

| | | | | | |
|---|---|---|---|---|---|
| H3018E05 | 4.90 | 1.93 | Krt2-8 | 16691 | keratin complex 2, basic, gene 8 |
| H3125F12 | 3.34 | 1.14 | Emd | 13726 | emerin |
| H3056C07 | 3.10 | 0.99 | Nup153 | 218210 | nucleoporin 153 |
| H3056C01 | 3.04 | 1.62 | Fmn2 | 54418 | formin 2 |
| H3139H08 | 2.29 | 1.42 | Ptma | 19231 | prothymosin alpha |
| H3119A03 | 2.14 | 1.86 | Syn1 | 20964 | synapsin I |

Down-regulated in response to S. pneumoniae at day 1
Cell division

| | | | | | |
|---|---|---|---|---|---|
| H3025F08 | 0.43 | 0.61 | Cul4b | 72584 | cullin 4B |
| H3027H03 | 0.49 | 0.73 | Rcbtb1 | 71330 | regulator of chromosome condensation (RCC1) and |
| | | | | | BTB (POZ) domain containing protein 1 |

Defense/immunity

| | | | | | |
|---|---|---|---|---|---|
| H3078D07 | 0.28 | 0.74 | Ier5 | 15939 | immediate early response 5 |
| H3127G03 | 0.44 | 0.83 | Samhd1 | 56045 | SAM domain and HD domain, 1 |
| H3026G05 | 0.44 | 1.10 | Tra1 | 22027 | tumor rejection antigen gp96 |
| H3027D05 | 0.48 | 0.88 | Ly6e | 17069 | lymphocyte antigen 6 complex, locus E |

Transcription

| | | | | | |
|---|---|---|---|---|---|
| H3129B04 | 0.27 | 0.54 | Dazap2 | 23994 | DAZ associated protein 2 |
| H3140H12 | 0.29 | 0.77 | Gtf2i | 14886 | general transcription factor II I |
| H3138G04 | 0.36 | 0.66 | Rpo2tc1 | 20024 | RNA polymerase II transcriptional coactivator |
| H3028C12 | 0.38 | 0.91 | Smarcad1 | 13990 | SWI/SNF-related, matrix-associated actin-dependent |
| | | | | | regulator of chromatin, subfamily a, containing DEAD/H |
| | | | | | box 1 |
| H3004F09 | 0.45 | 0.97 | Ssrp1 | 20833 | structure specific recognition protein 1 |
| H3127H11 | 0.47 | 0.87 | Ankfy1 | 11736 | ankyrin repeat and FYVE domain containing 1 |
| H3027B05 | 0.47 | 0.62 | Set | 56086 | SET translocation |
| H3117B06 | 0.48 | 0.84 | Mef2a | 17258 | myocyte enhancer factor 2A |
| H3127D06 | 0.49 | 0.87 | Hoxd9 | 15438 | homeo box D9 |
| H3027E01 | 0.49 | 0.60 | H2afz | 51788 | H2A histone family, member Z |
| H3123E02 | 0.49 | 0.59 | Cnbp | 12785 | cellular nucleic acid binding protein |

Translation/processing

| | | | | | |
|---|---|---|---|---|---|
| H3129E12 | 0.37 | 0.91 | Gorasp2 | 70231 | golgi reassembly stacking protein 2 |
| H3022H02 | 0.42 | 0.73 | Ctsl | 13039 | cathepsin L |
| H3032D10 | 0.44 | 0.69 | Eif5 | 217869 | eukaryotic translation initiation factor 5 |
| H3149H05 | 0.45 | 0.86 | Usp9x | 22284 | ubiquitin specific protease 9, X chromosome |
| H3025B02 | 0.48 | 1.33 | Psma7 | 26444 | proteasome (prosome, macropain) subunit, alpha type 7 |
| H3121H04 | 0.48 | 0.69 | Pes1 | 64934 | pescadillo homolog 1, containing BRCT domain |
| | | | | | (zebrafish) |
| H3107A02 | 0.49 | 0.94 | Hnrpk | 15387 | heterogeneous nuclear ribonucleoprotein K |
| H3004H04 | 0.49 | 0.67 | Ddx52 | 78394 | DEAD (Asp-Glu-Ala-Asp) box polypeptide 52 |
| H3126H02 | 0.49 | 0.61 | Snx5 | 69178 | sorting nexin 5 |
| H3030E10 | 0.50 | 0.85 | Ctsb | 13030 | cathepsin B |
| H3078B03 | 0.50 | 0.72 | Dnajb9 | 27362 | DnaJ (Hsp40) homolog, subfamily B, member 9 |
| H3114D10 | 0.50 | 0.71 | Rps3a | 20091 | ribosomal protein S3a |

RNA binding/splicing

| | | | | | |
|---|---|---|---|---|---|
| H3063D07 | 0.45 | 1.68 | Cpeb4 | 67579 | cytoplasmic polyadenylation element binding protein 4 |
| H3031H09 | 0.46 | 0.92 | Xrn2 | 24128 | 5′-3′ exoribonuclease 2 |
| H3004H03 | 0.47 | 0.74 | Snrpf | 69878 | small nuclear ribonucleoprotein polypeptide F |

Metabolism

| | | | | | |
|---|---|---|---|---|---|
| H3117G12 | 0.30 | 0.86 | Alox5ap | 11690 | arachidonate 5-lipoxygenase activating protein |
| H3126F02 | 0.43 | 0.77 | Cox6c | 12864 | cytochrome c oxidase, subunit VIc |
| H3142A08 | 0.43 | 0.75 | Uqcrc2 | 67003 | RIKubiquinol cytochrome c reductase core protein 2 |
| H3128H02 | 0.43 | 0.68 | Ldh2 | 16832 | lactate dehydrogenase 2, B chain |
| H3031F01 | 0.43 | 0.62 | Uqcrc1 | 22273 | ubiquinol-cytochrome c reductase core protein 1 |
| H3129F04 | 0.48 | 0.65 | Atp5f1 | 11950 | ATP synthase, H+ transporting, mitochondrial F0 |
| | | | | | complex, subunit b, isoform 1 |
| H3124F04 | 0.51 | 0.74 | Mgst1 | 56615 | microsomal glutathione S-transferase 1 |

Signal transduction

| | | | | | |
|---|---|---|---|---|---|
| H3146F01 | 0.36 | 0.54 | Arha | 11848 | ras homolog gene family, member A |
| H3146E11 | 0.40 | 0.53 | Rab7 | 19349 | RAB7, member RAS oncogene family |
| H3145H08 | 0.45 | 0.62 | Emr1 | 13733 | EGF-like module containing, mucin-like, hormone |
| | | | | | receptor-like sequence 1 |
| H3140F04 | 0.47 | 0.78 | Anxa6 | 11749 | annexin A6 |
| H3130C08 | 0.48 | 0.71 | Mapk3 | 26417 | mitogen activated protein kinase 3 |
| H3131D02 | 0.52 | 0.79 | Tnk2 | 51789 | tyrosine kinase, non-receptor, 2 |

Structure proteins

| | | | | | |
|---|---|---|---|---|---|
| H3129G04 | 0.15 | 0.58 | Vil2 | 22350 | villin 2 |
| H3015D01 | 0.47 | 0.75 | Tubb5 | 22154 | tubulin, beta 5 |
| H3036D07 | 0.52 | 1.15 | Catns | 12388 | catenin src |

^{a}PneD1 represents day 1 after infection of mice with Strptococcus pneumoniae.
^{b}Control represents uninfected control mice.
^{c}FluD1 represents day 1 after infection of mice with influenza virus A/PR/8/34.
^{d}LocusLink is superceded by Entrez Gene. LocusLink number is the same as GeneID and can be used to search gene database at the NIH web site: http:www.ncbi.nlm.nih.gov/query.fcgi?db=gene.

The 48 genes exclusively down-regulated in mice infected with S. pneumoniae but unchanged in mice infected with influenza virus A/PR/8/34 at day 1 were divided into 9 functional groups (Table 4). We identified 9 genes whose expression decreased more than 2.5-fold after infection with S. pneumoniae. These 9 genes are villin 2 gene (Vil2, down 6.7-fold), DAZ associated protein 2 gene (Dazap2, down 3.7-fold), immdediate early response 5 gene (Ier5, down 3.6-fold), general transcription factor II i gene (Gtf2i, down 3.4-fold), arachidonate 5-lipoxygenase activating protein gene (Alox5ap, down 3.3-fold), RNA polymerase II transcriptional coactivator gene (Rpo2tc1, down 2.8-fold), ras homolog gene family member A gene (Arha, down 2.8-fold), golgi reassembly stacking protein 2 gene (Gorasp2, down 2.7-fold), and SWI/SNF-related regulator of chromatin gene (Smarcad1, down 2.6-fold).

Identification of Genes Exclusively Up- or Down-regulated in Response to S. pneumoniae at Day 7

There were 21 known genes exclusively up-regulated in mice infected with S. pneumoniae but unchanged in mice infected with influenza virus A/PR/8/34 at day 7 (see Table 5 below).

TABLE 5

| | | | | | |
|---|---|---|---|---|---|
| | | | | Locus | |
| CloneID | PneD7^{a}/Ctr^{b} | FluD7^{c}/Ctr | Gene Name | Link^{d} | Gene Description |

Up-regulated in response to S. pneumoniae at day 7

| | | | | | |
|---|---|---|---|---|---|
| H3041F03 | 4.30 | 0.77 | Actb | 11461 | actin, beta, cytoplasmic |
| H3073G02 | 4.01 | 0.89 | Slc5a6 | 330064 | solute carrier family 5 (sodium-dependent vitamin |
| | | | | | transporter), member 6 |
| H3112E03 | 3.81 | 0.94 | Dlgh1 | 13383 | discs, large homolog 1 (Drosophila) |
| H3079A03 | 3.04 | 0.86 | Ccl6 | 20305 | chemokine (C—C motif) ligand 6 |
| H3001G08 | 2.91 | 0.88 | Rbm14 | 56275 | RNA binding motif protein 14 |
| H3009H03 | 2.74 | 0.72 | Slc16a6 | 104681 | solute carrier family 16 (monocarboxylic acid |
| | | | | | transporters), member 6 |
| H3053H09 | 2.59 | 1.05 | Axl | 26362 | AXL receptor tyrosine kinase |
| H3052D04 | 2.55 | 1.42 | Cox7a2 | 12866 | cytochrome c oxidase, subunit VIIa 2 |
| H3054E03 | 2.53 | 0.90 | Gsn | 227753 | gelsolin |
| H3079A06 | 2.461.77 | 1.77 | Eef1a1 | 13627 | eukaryotic translation elongation factor 1 alpha 1 |
| H3055D06 | 2.42 | 1.34 | Zfp239 | 22685 | zinc finger protein 239 |
| H3051A01 | 2.42 | 0.92 | Camk4 | 12326 | calcium/calmodulin-dependent protein kinase IV |
| H3109G08 | 2.39 | 1.07 | Ache | 11423 | acetylcholinesterase |
| H3020D07 | 2.33 | 1.66 | Rpl28 | 19943 | ribosomal protein L28 |
| H3129C02 | 2.31 | 1.32 | Eef2 | 13629 | eukaryotic translation elongation factor 2 |
| H3056B01 | 2.091.26 | 1.26 | Men1 | 17283 | multiple endocrine neoplasia 1 |
| H3053E07 | 2.09 | 0.99 | Als2cr2 | 227154 | amyotrophic lateral sclerosis 2 (juvenile) chromosome |
| | | | | | region, candidate 2 homolog (human) |
| H3032F06 | 2.08 | 1.24 | Cbx5 | 12419 | chromobox homolog 5 (Drosophila HP1a) |
| H3121H02 | 2.07 | 1.22 | Osgep | 66246 | O-sialoglycoprotein endopeptidase |
| H3064C12 | 2.04 | 1.28 | Smc6l1 | 67241 | SMC6 structural maintenance of chromosomes 6-like 1 |
| | | | | | (yeast) |
| H3131G04 | 2.04 | 0.71 | Ddx5 | 13207 | DEAD (Asp-Glu-Ala-Asp) box polypeptide 5 |

Down-regulated in response to S. pneumoniae at day 7

| | | | | | |
|---|---|---|---|---|---|
| H3117D06 | 0.34 | 0.80 | Rpl36 | 54217 | ribosomal protein L36 |
| H3040B10 | 0.36 | 1.16 | Bing4 | 57315 | BING4 protein |
| H3087A12 | 0.40 | 0.80 | Ltf | 17002 | lactotransferrin |
| H3111H07 | 0.44 | 1.02 | Lypla1 | 18777 | lysophospholipase 1 |
| H3115A05 | 0.46 | 0.59 | Plcd | 18799 | phospholipase C, delta |
| H3002H11 | 0.47 | 0.66 | Calb1 | 12307 | calbindin-28K |
| H3134G11 | 0.48 | 0.71 | Rpl37a | 19981 | ribosomal protein L37a |
| H3120B03 | 0.48 | 0.72 | Rps26 | 27370 | ribosomal protein S26 |
| H3113B08 | 0.49 | 0.69 | Atp5k | 11958 | ATP synthase, H+ transporting, mitochondrial F1F0 |
| | | | | | complex, subunit e |
| H3097E03 | 0.49 | 0.82 | Cbr2 | 12409 | carbonyl reductase 2 |
| H3114A05 | 0.52 | 0.67 | Gpx4 | 14779 | glutathione peroxidase 4 |

^{a}PneD7 represents day 7 after infection of mice with Strptococcus pneumoniae.
^{b}Control represents uninfected control mice.
^{c}FluD7 represents day 7 after infection of mice with influenza virus A/PR/8/34.
^{d}LocusLink is superceded by Entrez Gene. LocusLink number is the same as GeneID and can be used to search gene database at the NIH web site: http:www.ncbi.nlm.nih.gov/query.fcgi?db=gene.

We identified 4 genes whose expression increased more than 3-fold after infection with S. pneumoniae: cytoplasmic beta actin gene (Atcb, up 4.3-fold), solute carrier family 5 member 6 gene (Slc5a6, up 4-fold), discs large homolog 1 gene (Dlgh1, up 3.8-fold) and chemokine (C-C motif) ligand 6 gene (Cc16, up 3-fold). There were 12 genes exclusively down-regulated at day 7 after infection of mice with S. pneumoniae but unchanged in mice infected with influenza virus A/PR/8/34 (Table 5). These 12 genes include ribosomal protein L36 gene (Rpl36, down 2.9-fold), BING4 protein gene (Bing4, down 2.8-fold), lactotransferrin gene (Ltf, down 2.5-fold), lysophospholipase 1 gene (Lyp1a1, down 2.3-fold), phospholipase C delta (Plcd, down 2.2-fold), calbindin gene (Calb1, down 2.1-fold) and glutathione peroxidase 4 gene (Gpx4, down 1.9-fold).

The genes exclusively up-regulated or down-regulated in mice infected with influenza virus A/PR/8/34 or S. pneumoniae are good candidate genes that could be used to distinguish mice exposed to specific classes of pathogenic agents such as influenza virus A/PR/8/34 and S. pneumoniae if their expression levels could be validated.

Identification of Genes Up- or Down-regulated in Response to Both Influenza Virus A/PR/8/34 and S. pneumoniae

As shown in Table 6 below, 15 known genes were up-regulated and 12 known genes down-regulated at day 1 (Table 6) in response to both influenza virus A/PR/8/34 and S. pneumoniae. Among the up-regulated genes in response to both pathogens at day 1 are hemoglobin alpha adult chain 1 gene (Hba-a1, up >3.1-fold), tyrosine 3-monooxygenase/trptophan 5-monooxygenase activation protein gene (Ywhaq, up >2.1-fold), UDP-glucose dehydrogenase (Ugdh, >2.4-fold), chorionic somatomanimotropin hormone 1 gene (Csh1, up >2.3-fold), cytoplasmic actin beta gene (Actb, up >2.5-fold), FGF receptor activating protein 1 gene (Frag1, up >2.4-fold), transmembrane channel-like gene family 6 gene (Tmc6, up >2-fold), cask-interacting protein 2 gene (Caskin2, up >2-fold), and NADH dehydrogenase 1 beta subcomplex 3 (Ndufb3, up >2-fold). The genes down-regulated in response to both pathogens at day 1 include thioredoxin interacting protein gene (Txnip, down >3-fold), ribosomal protein L32 gene (Rp132, down >1.9-fold), block of proliferation 1 gene (Bop1, down >2-fold), cyclin D binding myb-like transcription factor 1 gene (Dmtf1, down >2-fold), attractin gene (Atrn, down >2.2-fold), ribosomal protein S19 gene (Rps19, down >2.2-fold), cyclin I gene (Ccni, down >1.96-fold), pyruvate kinase gene (Pkm2, down >1.92-fold) and putative serine/threonine kinase gene (Prkx, down >1.96-fold).

TABLE 6

| | | | | |
|---|---|---|---|---|
| | FluD1^{a}/ | | Locus | |
| CloneID | Ctr^{b} | PneD1^{c}/Ctr | Link^{d} | Gene Description |

Gene Name

Up-regulated in response to both influenza virus A and S. pneumoniae at day 1

| | | | | | |
|---|---|---|---|---|---|
| H3122H10 | 5.11 | 3.19 | Hba-a1 | 15122 | hemoglobin alpha, adult chain 1 |
| H3120H10 | 3.79 | 2.19 | Ywhaq | 22630 | tyrosine 3-monooxygenase/tryptophan 5- |
| | | | | | monooxygenase |
| | | | | | activation protein, theta polypeptide |
| H3002G08 | 3.70 | 4.43 | Tex292 | 21771 | testis expressed gene 292 |
| H3045C05 | 3.65 | 2.40 | Ugdh | 22235 | UDP-glucose dehydrogenase |
| H3011H02 | 3.49 | 2.26 | Csh1 | 18775 | chorionic somatomammotropin hormone 1 |
| H3122H11 | 3.47 | 2.70 | Hba-a1 | 15122 | hemoglobin alpha, adult chain 1 |
| H3011G02 | 2.67 | 2.47 | Actb | 11461 | actin, beta, cytoplasmic |
| H3036E12 | 2.41 | 2.92 | Frag1-pending | 233575 | FGF receptor activating protein 1 |
| H3115A04 | 2.38 | 2.73 | Snrp116- | 20624 | U5 small nuclear ribonucleoprotein |
| | | | pending | | |
| H3090H06 | 2.38 | 2.09 | Tmc6 | 217353 | transmembrane channel-like gene family 6 |
| H3106B07 | 2.22 | 2.42 | Nr2e1 | 21907 | nuclear receptor subfamily 2, group E, member 1 |
| H3016G08 | 2.19 | 2.06 | Caskin2 | 140721 | cask-interacting protein 2 |
| H3043E11 | 2.15 | 2.38 | CGI-09- | 66926 | CGI-09 protein |
| | | | pending | | |
| H3012B07 | 2.11 | 2.30 | Impdh2 | 23918 | inosine 5′-phosphate dehydrogenase 2 |
| H3011C09 | 2.10 | 2.06 | Ndufb3 | 66495 | NADH dehydrogenase (ubiquinone) 1 beta subcomplex 3 |
| H3011C10 | 2.08 | 2.37 | Rpl5 | 19983 | ribosomal protein L5 |

Down-regulated in response to both influenza virus A and S. pneumoniae at day 1

| | | | | | |
|---|---|---|---|---|---|
| H3128D02 | 0.22 | 0.33 | Txnip | 56338 | thioredoxin interacting protein |
| H3148E05 | 0.32 | 0.51 | Rpl32 | 19951 | ribosomal protein L32 |
| H3118D01 | 0.39 | 0.47 | Bop1 | 12181 | block of proliferation 1 |
| H3144H08 | 0.40 | 0.48 | Dmtf1 | 23857 | cyclin D binding myb-like transcription factor 1 |
| H3118H02 | 0.42 | 0.38 | Plagl1 | 22634 | pleiomorphic adenoma gene-like 1 |
| H3145D08 | 0.43 | 0.33 | Sco1 | 52892 | SCO cytochrome oxidase deficient homolog 1 |
| | | | | | (yeast) |
| H3115A02 | 0.43 | 0.45 | Atrn | 11990 | attractin |
| H3146B07 | 0.44 | 0.43 | Rps19 | 20085 | ribosomal protein S19 |
| H3149G05 | 0.45 | 0.51 | Ccni | 12453 | cyclin I |
| H3030D10 | 0.47 | 0.52 | Pkm2 | 18746 | pyruvate kinase, muscle |
| H3149A02 | 0.49 | 0.41 | Brpf1 | 78783 | bromodomain and PHD finger containing, 1 |
| H3136F06 | 0.51 | 0.44 | Prkx | 19108 | putative serine/threonine kinase |

Up-regulated in response to both influenza virus A and S. pneumoniae at day 7

| | | | | | |
|---|---|---|---|---|---|
| H3024G04 | 3.87 | 4.11 | Defcr-rs1 | 13218 | defensin related sequence cryptdin peptide (paneth |
| | | | | | cells) |
| H3126A06 | 2.60 | 3.76 | Eef1a1 | 13627 | eukaryotic translation elongation factor 1 alpha 1 |
| H3136A11 | 2.27 | 3.56 | Psmb4 | 19172 | proteasome (prosome, macropain) subunit, beta |
| | | | | | type 4 |
| H3118H01 | 2.20 | 2.09 | D3Jfr1 | 229663 | DNA segment, Chr 3, MJeffers 1 |
| H3010D02 | 2.10 | 3.08 | Actb | 11461 | actin, beta, cytoplasmic |

Name

Down-regulated in response to both influenza virus A and S. pneumoniae at day 7

| | | | | | |
|---|---|---|---|---|---|
| H3002D08 | 0.48 | 0.54 | Abcb8 | 27417 | ATP-binding cassette, sub-family B (MDR/TAP), |
| | | | | | member 8 |
| H3129F04 | 0.50 | 0.53 | Atp5f1 | 11950 | ATP synthase, H+ transporting, mitochondrial F0 |
| | | | | | complex, subunit b, isoform 1 |

^{a}FluD1 represents day 1 after infection of mice with influenza virus A/PR/8/34.
^{b}Control represents uninfected control mice.
^{d}LocusLink is superceded by Entrez Gene. LocusLink number is the same as GeneID and can be used to search gene database at the NIH web site: http:www.ncbi.nlm.nih.gov/query.fcgi?db=gene.
^{c}PneD1 represents day 1 after infection of mice with Strptococcus pneumoniae.
^{e}FluD7 represents day 7 after infection of mice with influenza virus A/PR/8/34.
^{f}PneD7 represents day 7 after infection of mice with Strptococcus pneumoniae.

Only 5 known genes were up-regulated and 2 known genes were down-regulated at day 7 (Table 6) in response to both influenza virus A/PR/8/34 and S. pneumoniae. These genes are defensin related sequence cryptdin peptide gene (Defcr-rs1, up >3.87-fold), eukaryotic translation elongation factor 1 alpha 1 gene (Eef1a1, up >2.6-fold), proteasome subunit beta type 4 gene (Psmb4, up >2.3-fold), cytoplasmic actin beta gene (Actb, up >2.1-fold), ATP synthase gene (Atp5f1, down >1.8-fold) and ATP-binding cassette sub-family B member 8 gene (Abcb8, down >1.8-fold).

Verification of differentially expressed genes by quantitative real-time RT-PCR. We sought to verify the microarray results by assessing the expression of a sample of 8 genes using quantitative real-time RT-PCR. We performed quantitative real-time RT-PCR with TaqMan chemistry using purified total RNAs from spleens of mice (day 1) infected with influenza virus A and S. pneumoniae as templates. Table 7 (below) compares the results obtained by microarray analysis and real-time RT-PCR. Results from quantitative Real-time RT-PCR confirmed the results obtained by cDNA microarray analysis in 5 out of 8 genes tested. The results showed that the expression changes for 5 of 8 selected genes were consistent with the direction of change predicted by microarray analysis. The magnitude of change determined by microarray and real-time RT-PCR were somewhat different, a result which was not surprising considering the technical differences in the methods of analysis and normalization.

TABLE 7

| | Relative Expression Levels | | | |
|---|---|---|---|---|
| | FluD1^{a}/Control^{b} | | PneD1^{c}/Control^{b} | |
| Gene | | Real-Time | | Real-Time |
| Name | Microarray | RT-PCR | Microarray | RT-PCR |
| Chi3I3 | 6.31 | 7.93 | 0.92 | 2.38 |
| Gle1I | 26.65 | 9.49 | 0.73 | 0.91 |
| Fen1 | 5.1 | 6.64 | 1 | 5.02 |
| Scamp1 | 0.92 | 0.94 | 5.57 | 6.16 |
| Gnai2 | 0.4 | 0.55 | 0.75 | 1.45 |
| Ier5 | 0.74 | 0.67 | 0.28 | 3.07 |
| Bop1 | 0.39 | 4.03 | 0.47 | 3.64 |
| Cpsf2 | 2.18 | 0.76 | 1.68 | 1.06 |

^{a}FluD1 represents day 1 after infection of mice with influenza virus A/PR/8/34.
^{b}Control represents uninfected control mice.
^{c}PneD1 represents day 1 after infection of mice with Strptococcus pneumoniae.

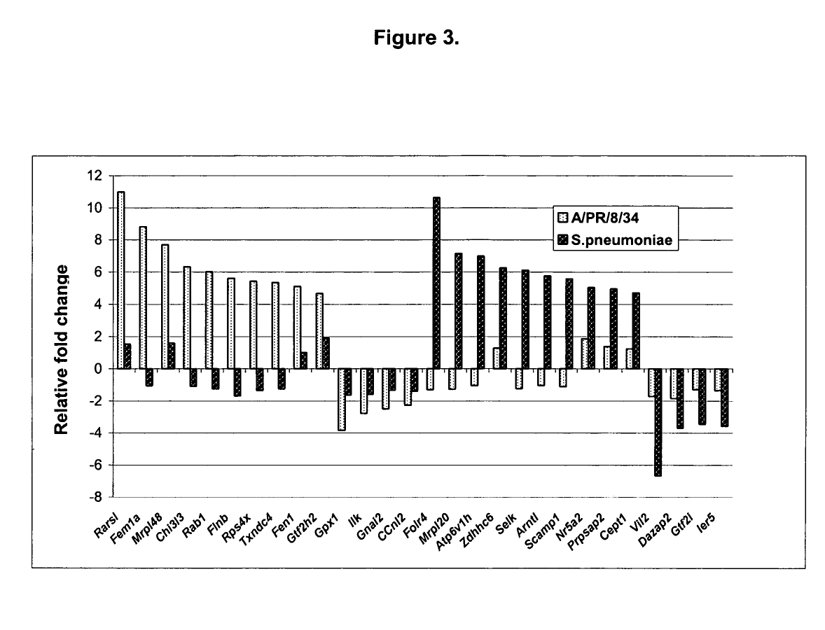

Signature gene expression patterns at day 1 after infection of mice. Based the microarray profiles of mice in response to influenza virus A/PR/8/34 and S. pneumoniae at day 1 after infection, signature gene expression patterns have been revealed by comparing relative fold changes of 28 differentially expressed genes (FIG. 3 and FIG. 11). These 28 differentially expressed genes are: Rars1, Fem1a, Mrp148, Chi313, Rab1, Flnb, Rps4x, Txndc4, Fen1, Gtf2h2, Gpx1, Ilk, Gnai2, Ccn12, Fo1r4, Mrp120, Atp6v1h, Zdhhc6, Selk, Arnt1, Scamp1, Nr5a2, Prpsap2, Cept1, Vil2, Dazap2, Gtf2i, and Ier5. The results show the clear difference between mice infected with influenza virus A/PR/8/34 and S. pneumoniae (FIG. 3). These selected genes are good candidate biomarkers for distinguishing mice exposed to specific microbial pathogens such as influenza virus A and S. pneumoniae. The identification of mouse model biomarkers for respiratory diseases is an important step towards identification of human biomarkers that could eventually lead to tests for early diagnosis of respiratory infections. The present invention provides a list of candidate biomarkers for use in human diagnosis. Results from this study demonstrate that microarray data from infected mice are useful tools for distinguishing between different classes of pathogenic agents.

We claim:

1. A method of assessing whether a subject is infected with an influenza A virus or Streptococcus pneumoniae (S. pneumoniae), the method comprising comparing:
(a) the level of expression of an influenza A infection marker or a S. pneumoniae infection marker in a subject sample, wherein the influenza A infection marker is selected from the group of markers listed in Tables 2 or 3, and wherein the S. pneumoniae infection marker is selected from the group of markers listed in Tables 4 or 5; and
(b) the normal level of expression of the influenza A infection marker or the S. pneumonia infection marker in a control sample,
wherein a detectable difference in the level of expression of the influenza A or S. pneumoniae infection marker in the subject sample and the normal level of expression of the influenza A or S. pneumoniae infection marker is an indication that the subject is infected with influenza A virus or S. pneumonaie.

2. The method of claim 1, wherein the influenza A or S. pneumoniae infection marker is a host immune response gene.

3. The method of claim 1, wherein the subject sample is obtained from the blood, lymphoid tissue, spleen or lung of the subject.

4. The method of claim 3, wherein the subject sample is whole blood, plasma, serum, or a nucleic acid molecule isolated or prepared from the blood.

5. The method of claim 1, wherein the level of expression of the influenza A or S. pneumoniae infection marker is determined by microarray analysis or real-time quantitative RT-PCR.

6. The method of claim 5, wherein the microarray analysis or real-time quantitative RT-PCR comprises hybridization of a nucleic acid molecule isolated or prepared from the subject sample and the influenza A or S. pneumoniae infection marker.

7. The method of claim 6, wherein the nucleic acid molecule or the influenza A or S. pneumoniae infection marker is a complementary DNA (cDNA) molecule.

8. The method of claim 7, wherein the cDNA molecule is between 15 and 40 nucleotides in length.

9. The method of claim 1, wherein the level of expression of the influenza A or S. pneumoniae infection marker in the subject sample is significantly different from the level of expression in the control sample.

10. A method of assessing whether a subject is infected with influenza A virus or S. pneumoniae, the method comprising comparing:
(a) the level of expression of an influenza A infection marker or a S. pneumoniae infection marker in a subject sample; and
(b) the normal level of expression of the influenza A infection marker or the S. pneumoniae infection marker in a control sample,
wherein the influenza A infection marker has a nucleotide sequence corresponding to an influenza marker gene listed in Tables 2 or 3 and the S. pneumoniae infection marker has a nucleotide sequence corresponding to an S. pneumoniae marker gene listed in Tables 4 or 5, and wherein a detectable difference in the level of expression of the influenza A or S. pneumoniae infection marker in the subject sample and the normal level of expression in the control sample is an indication that the subject is infected with influenza A virus or S. pneumoniae.

11. The method of claim 10, wherein said the influenza A and S. pneumoniae infection markers are human immune response genes.

12. The method of claim 10, wherein the level of expression of the influenza A or S. pneumoniae, infection marker is determined by microarray analysis or real-time quantitative RT-PCR.

13. A method of identifying a marker of infection by influenza A virus or S. pneumonia, comprising the steps of:
(a) obtaining a sample from a subject that is infected with an influenza A virus or S. pneumonia;
(b) isolating and labeling mRNA from the sample;
(c) detecting labeled mRNA from the sample to produce a gene expression profile; and
(d) comparing the gene expression profile from step (c) with a reference gene expression profile of a control to determine a difference in the level of expression of at least one gene;
wherein the difference in level of expression is determined by microarray analysis or real-time quantitative RT-PCR, and wherein the difference in the level of expression of the at least one gene identifies the gene as a marker of infection by influenza A virus or S. pneumonia.

14. The method of claim 13, wherein the influenza A or S. pneumoniae infection marker is a host immune response gene.

15. The method of claim 13, wherein the microarray analysis or real-time quantitative RT-PCR comprises hybridization of a nucleic acid molecule isolated or prepared from a sample obtained from a non-human animal and the influenza A or S. pneumoniae infection marker.

16. The method of claim 13, wherein the subject is a human or non-human animal.

17. The method of claim 16, wherein the non-human animal is a mouse, and wherein at least one gene is a marker gene selected from at least one of Tables 2-6.

18. A gene expression profile comprising at least one gene identified by the method of claim 13.

19. A kit for diagnosing an influenza A infection or an S. pneumoniae infection in a subject, the kit comprising at least one gene identified by the method of claim 13.

20. A kit for diagnosing an influenza A and S. pneumoniae infection in a subject, the kit comprising at least one gene identified by the method of claim 13.


SEQUENCE LISTING (27 sequences)

SEQ ID NO: 1 - DNA, 18 bp, Mouse Gnai2-F
ggtgctggctgaggatga

SEQ ID NO: 2 - DNA, 20 bp, Mouse Gnai2-R
tccttcttgttgaggaagag

SEQ ID NO: 3 - DNA, 23 bp, Mouse Gnai2-P
catgcatgagagcatgaagctgt

SEQ ID NO: 4 - DNA, 21 bp, Mouse Glel-F
ttcatcaataccactcaggca

SEQ ID NO: 5 - DNA, 21 bp, Mouse Gle-R
ggagtgtacatgactttgtac

SEQ ID NO: 6 - DNA, 23 bp, Mouse Gle-P
ctgtcaccattgaaggaccatcc

SEQ ID NO: 7 - DNA, 21 bp, Mouse Chi3-F
tgagtgacccttctaagactg

SEQ ID NO: 8 - DNA, 22 bp, Mouse Chi3-R
ggctccttcattcagaaatgta

SEQ ID NO: 9 - DNA, 23 bp, Mouse Chi3-P
agtactggcccaccaggaaagta

SEQ ID NO: 10 - DNA, 18 bp, Mouse Scam-F
ggtgctggctgaggatga

SEQ ID NO: 11 - DNA, 20 bp, Mouse Scam-R
tccttcttgttgaggaagag

SEQ ID NO: 12 - DNA, 23 bp, Mouse Scam-P
catgcatgagagcatgaagctgt

SEQ ID NO: 13 - DNA, 18 bp, Mouse Ler5-F
ggtgctggctgaggatga

SEQ ID NO: 14 - DNA, 20 bp, Mouse Ler5-R
tccttcttgttgaggaagag

SEQ ID NO: 15 - DNA, 23 bp, Mouse Ler5-P
catgcatgagagcatgaagctgt

SEQ ID NO: 16 - DNA, 18 bp, Mouse Bobl-F
ggtgctggctgaggatga

SEQ ID NO: 17 - DNA, 20 bp, Mouse Bobl-R
tccttcttgttgaggaagag

SEQ ID NO: 18 - DNA, 23 bp, Mouse Bobl-P
catgcatgagagcatgaagctgt

SEQ ID NO: 19 - DNA, 18 bp, Mouse Cpsf2-F
ggtgctggctgaggatga

SEQ ID NO: 20 - DNA, 20 bp, Mouse Cpsf2-R
tccttcttgttgaggaagag

SEQ ID NO: 21 - DNA, 23 bp, Mouse Cpsf2-P
catgcatgagagcatgaagctgt

SEQ ID NO: 22 - DNA, 20 bp, Mouse Fenl-F
ccagagaactggctccacaa

SEQ ID NO: 23 - DNA, 19 bp, Mouse Fenl-R
tggctcgctccacttcagc

SEQ ID NO: 24 - DNA, 23 bp, Mouse Fenl-P
cttcctggagccagaagtactgg

SEQ ID NO: 25 - DNA, 21 bp, Mouse AfG-F
ccaccaccaagacctactttc

SEQ ID NO: 26 - DNA, 19 bp, Mouse AfG-R
gcatgcaggtcgctcagag

SEQ ID NO: 27 - DNA, 21 bp, Mouse AfG-P
agccacggctctgcccaggtc